(19) United States
(12) Patent Application Publication
(10) Pub. No.: US 2022/0079194 A1
(43) Pub. Date: Mar. 17, 2022

(54) EXTRUDATE FOOD COMPOSITIONS COMPRISING CULTIVATED ANIMAL CELLS AND METHODS OF PRODUCTION THEREOF

(71) Applicant: GOOD Meat, Inc., San Francisco, CA (US)

(72) Inventors: Meng LI, San Francisco, CA (US); Nathaniel PARK, San Francisco, CA (US); Vitor Espirito SANTO, San Francisco, CA (US)

(21) Appl. No.: 17/460,083

(22) Filed: Aug. 27, 2021

Related U.S. Application Data
(60) Provisional application No. 63/071,806, filed on Aug. 28, 2020.

(51) Int. Cl. A23L 13/40; C12N 5/077; B33Y 70/10; B33Y 80/00; A23J 3/04; A23J 3/14; A23L 29/00; A23P 30/20; C12N 9/10

(52) U.S. Cl. CPC A23L 13/43; C12Y 203/02013; B33Y 70/10; B33Y 80/00; A23J 3/04; A23J 3/14; A23L 13/426; A23L 13/48; A23L 29/06; A23P 30/20; C12N 9/1044; C12N 2500/90; C12N 2501/115; C12N 2501/11; C12N 5/0652

(57) ABSTRACT

Provided herein are extruded food products comprising cultivated animal cells and a plant protein. Also provided are methods for preparing extruded food products.

### FIELD

The present disclosure relates to extruded food products comprising cultivated animal cells and methods of producing extruded food products.

### BACKGROUND

Meat from farmed animals has been a part of the human diet for thousands of years. Poultry, beef, pork and other animals are consumed the world over and it is well recognized that the farming of animals contributes significantly to global warming. In 2006, the United Nations Food and Agricultural Organization estimated that animal farming produces about 18 percent of the total greenhouse gases produced by human activity. The UN estimated that greenhouse gases produced by animal farming exceeded greenhouse gases produced by the entire transportation industry, including greenhouse gases produced by automobiles, trucks, trains, ships, and airplanes combined.

Additionally, there are health risks in consuming farmed animals. The slaughter and processing of animals exposes the animal carcasses to microbial contamination and exposes people to potentially deadly microbes that remain on the meat. In random surveys of chicken products across the United States in 2012, the Physicians Committee for Responsible Medicine found 48% of samples to contain fecal matter, and a 2009 USDA study found that 87% of chicken carcasses tested positive for generic E. coli, a sign of fecal contamination, just prior to packaging. While thorough cooking can kill contaminating microorganisms, if cooking is not thorough, some microorganisms may survive to cause foodborne illness.

Use of extrusion cooking to make meat analogue products can be dated to the 1960s. However, current existing technologies all use common starch and proteins derived from plants, such as soy, wheat, potato, or rice. Conventional methods and processes used for producing meat analogues using only plant-based resources provide limited nutritional value and organoleptic performance when compared to products that contain cultivated animal cells.

Cultured meat products have the potential to: (1) substantially reduce reliance on slaughtered animals for food use, (2) lessen the environmental burden of raising animals for food supply, and (3) provide a reliable source of protein that is both safe and has consistent quality.

Extruded food products made with cultivated animal cells produce foods that have superior organoleptic performance and better nutritional profile as compared to meat analogues that use only plant proteins.

### SUMMARY

The present disclosure provides an extrudate comprising cultivated animal cells, a plant protein, and at least one other ingredient.

The cultivated animal cell is cultivated in vitro in growth media with or without animal serum.

In some embodiments, the cultivated animal cell is an avian cell, a bovine cell, a porcine cell or a fish cell.

In some embodiments, the extrudate comprises a plant protein. In some embodiments, the plant protein is a plant protein isolate or a plant protein concentrate.

In some embodiments, the extruded product optionally comprises a peptide cross-linking agent.

In some embodiments, the at least one other ingredient in the extrudate in some embodiments is lipid, salt, sugar, fiber, humectant, flavorant, colorant, and/or preservative.

In some embodiments, the salt is selected from the group consisting of disodium phosphate, sodium hexametaphosphate, sodium citrate, sodium chloride, sodium sulfate, sodium acetate, sodium diacetate, sodium phosphate, potassium chloride, potassium sulfate, or potassium phosphate, calcium citrate, calcium chloride, magnesium citrate, and magnesium chloride.

In some embodiments, the extrudate has a fibrous structure.

In some embodiments, the extrudate has a Wamer-Bratzler score of between 5 N to 300 N.

In some embodiments, the extrudate is a scaffold.

In some embodiments, the extrudate is a substrate for 3D printing.

A method of preparing an extrudate comprising a plant protein, at least one other ingredient, and optionally, a peptide cross-linking enzyme is provided. The method comprises preparing a dough by contacting water and a plant protein. The dough is placed into the hopper of an extrusion machine. The dough is transferred from the hopper into the barrel of an extrusion machine and conveyed through the barrel under mechanical pressure. Optionally, the dough can be heated during conveyance of the dough through the barrel. The dough can be heated by injecting steam into the barrel or by heating the barrel. During the conveyance of the dough through the barrel, the cultivated animal cells or cell paste is injected into the barrel to produce a dough/cell admixture. Next, the dough/cell admixture is extruded through a die to produce the extrudate. In some embodiments, the at least one other ingredient can be added to the dough prior to placing the dough into the hopper. In some embodiments, the at least one other ingredient is injected into the barrel of the extruder before, during, after or together with the injection of the cultivated animal cells into the barrel.

A method of preparing an extrudate comprising cultivated animal cells, a plant protein, at least one other ingredient, and optionally, a peptide cross-linking agent is provided. The method comprises contacting water, plant protein and cultivated animal cells to produce a dough/cell admixture. The dough/cell admixture is placed into the hopper of an extrusion machine. The dough/cell admixture is transferred from the hopper into the barrel of an extrusion machine. The dough/cell admixture is conveyed through the barrel under mechanical pressure. Optionally, the dough/cell admixture is heated during the conveyancing through the barrel. The dough/cell admixture is extruded through a die to produce the extrudate. In some embodiments, the at least one other ingredient is incorporated into the dough/cell admixture prior to placing the dough/cell admixture into the hopper. In some embodiments, the at least one other ingredient is injected into the barrel of the extruder during the conveyance of the dough/cell admixture through the barrel.

In some embodiments, the heating of the dough or the dough/cell admixture is accomplished by injecting steam into the barrel or by heating the barrel.

In some embodiments, the extrusion method is a wet extrusion method or a dry extrusion method.

The extrusion machine is in some embodiments a single screw extrusion machine or in other embodiments, a double screw extrusion machine.

In some embodiments of the methods, the peptide cross-linking enzyme is contacted with the dough. In other embodiments, the peptide cross-linking enzyme is contacted with the dough/cell admixture. In other embodiments, the peptide cross-linking enzyme is contacted with the dough and the dough/cell admixture.

The present disclosure provides methods for culturing avian fibroblast cells in vitro for use in preparing extruded food products. This disclosure also sets forth processes for making and using products.

In some embodiments, there are provided methods of producing a food product comprising animal cells. In some embodiments, the animal cells are muscle cells, fat cells, fibroblast cells, epithelial cells, osteoblasts, chondrocytes, connective tissue cells, hematopoietic cells, induced pluripotent stem cells, isolated pluripotent and multipotent stem cells, induced or isolated stem cells that are differentiated into various cell types including muscle cells, fat cells, fibroblast cells, epithelial cells, osteoblasts, chondrocytes, connective tissue cells, hematopoietic cells and other cell types.

In some embodiments, there are provided methods of producing a food product comprising avian fibroblast cells cultured in vitro, the methods comprising culturing a population of avian fibroblast cells in vitro in a growth medium capable of maintaining the avian fibroblast cells, recovering the avian fibroblast cells, and formulating the recovered avian fibroblast cells into an edible food product by extrusion. In some embodiments, the avian fibroblast cells comprise primary avian fibroblast cells. In some embodiments, the avian fibroblast cells comprise secondary avian fibroblast cells. In some embodiments, the avian fibroblast cells are fibroblasts differentiated in vitro.

In some embodiments, there are provided methods of producing a food product comprising avian muscle cells, fat cells, fibroblast cells, epithelial cells, osteoblasts, chondrocytes, connective tissue cells, hematopoietic cells, induced pluripotent stem cells, isolated pluripotent and multipotent stem cells, induced or isolated stem cells that are differentiated into various cell types including muscle cells, fat cells, fibroblast cells, epithelial cells, osteoblasts, chondrocytes, connective tissue cells, hematopoietic cells and other avian cell types cultured in vitro, the methods comprising culturing a population of avian cells in vitro in a growth medium capable of maintaining the avian cells, recovering the cells, and formulating the recovered avian cells into an edible food product by extrusion. In some embodiments, the avian cells comprise primary cells. In some embodiments, the avian cells comprise secondary cells. In some embodiments, the avian cells are differentiated in vitro to the desired avian cell types.

In some embodiments, there are provided methods of producing a food product comprising bovine muscle cells, fat cells, fibroblast cells, epithelial cells, osteoblasts, chondrocytes, connective tissue cells, hematopoietic cells, induced pluripotent stem cells, isolated pluripotent and multipotent stem cells, induced or isolated stem cells that are differentiated into various cell types including muscle cells, fat cells, fibroblast cells, epithelial cells, osteoblasts, chondrocytes, connective tissue cells, hematopoietic cells and other avian cell types cultured in vitro, the methods comprising culturing a population of bovine cells in vitro in a growth medium capable of maintaining the bovine cells, recovering the cells, and formulating the recovered bovine cells into an edible food product by extrusion. In some embodiments, the bovine cells comprise primary cells. In some embodiments, the bovine cells comprise secondary cells. In some embodiments, the bovine cells are differentiated in vitro to the desired avian cell types

In some embodiments, there are provided methods of preparing a food product made from avian fibroblast cells grown in vitro, the method comprising the steps of: conditioning water with a phosphate to prepare conditioned water, hydrating a plant protein isolate or plant protein concentrate with the conditioned water to produce hydrated plant protein, contacting the cell paste with the hydrated plant protein to produce a cell and pulse protein mixture, heating the cell and plant protein mixture in steps, wherein the steps comprise at least one of: ramping up the temperature of the cell and protein mixture to a temperature between 40-65° C., maintaining the temperature of the cell and protein mixture at a temperature between 40-65° C. for 1 to 30 minutes, ramping up the temperature of the cell and protein mixture to a temperature between 60-85° C., cooling the cell and protein mixture to a temperature between −1-25° C., and admixing the cell and protein mixture with a fat to create a pre-cooking product. The pre-cooking product can be consumed without further cooking. Alternatively, the pre-cooking product is cooked to produce the edible food product. Optionally, the pre-cooking product may be stored at room temperature, refrigeration temperatures or frozen.

In some embodiments, there are provided food products produced from avian fibroblasts, comprising a cell paste, the cell paste content of at least 5% by weight, and wherein the cell paste is made from avian fibroblast cells grown in vitro; a plant protein isolate or plant protein concentrate, the plant protein content at least 5% by weight; a fat, the fat content at least 5% by weight; and water, the water content at least 5% by weight.

In some embodiments, the food composition or food product comprises about 1%-100% by weight wet cell paste.

In some embodiments, plant protein isolates or plant protein concentrates are obtained from pulses selected from the group consisting of dry beans, lentils, mung beans, fava beans, dry peas, chickpeas, cowpeas, bambara beans, pigeon peas, lupins, vetches, adzuki, common beans, fenugreek, long beans, lima beans, runner beans, tepary beans, soy beans, or mucuna beans. In various embodiments, the pulse protein isolates or plant protein concentrates provided herein are derived from Vigna angularis, Vicia faba, Cicer arietinum, Lens culinaris, Phaseolus vulgaris, Vigna unguiculata, Vigna subterranea, Cajanus cajan, Lupinus sp., Vetch sp., Trigonella foenum-graecum, Phaseolus lunatus, Phaseolus coccineus, or Phaseolus acutifolius. In some embodiments, the pulse protein isolates are derived from mung beans. In some embodiments, the mung bean is Vigna radiata.

In some embodiments, animal protein isolate and animal protein concentrate are obtained from animals or animal products. Examples of animal protein isolate or animal protein concentrate include whey, casein, and egg protein.

In some embodiments, plant protein isolates are obtained from wheat, rice, teff, oat, corn, barley, sorghum, rye, millet, triticale, amaranth, buckwheat, quinoa, almond, cashew, pecan, peanut, walnut, macadamia, hazelnut, pistachio, brazil, chestnut, kola nut, sunflower seeds, pumpkin seeds, flax seeds, cacao, pine nut, ginkgo, or other nuts.

In some embodiments, a cell paste comprising cultivated animal cells is provided. When the cell density of the wet cell paste is between 1×10⁹ to 100×10⁹ cells per ml, at a temperature of between 30° C. and 95° C., the storage modulus (G′) is between 5 Pa and 100 Pa.

### BRIEF DESCRIPTIONS OF THE DRAWINGS

FIG. 1 depicts a process diagram for culturing of avian fibroblast cells.

FIG. 2 depicts a process diagram for harvesting cultured avian fibroblast cells.

FIG. 3 depicts a hierarchical clustering of the transcriptome analysis of three biological replicates of chicken cell pools (JUST1, JUST2, JUST3) used to manufacture a cultured chicken meat product (JUST7, JUST8, JUST9).

FIG. 4A depicts chicken fibroblast cell adaptation in low serum media indicating cell viability as a function of culture time. FIG. 4B depicts chicken fibroblast cell adaptation in low serum media indicating population doubling time as a function of passage number.

FIG. 5A depicts chicken fibroblast cell adaptation in basal media supplemented with fatty acids and growth factors as a function of culture time. FIG. 5B depicts chicken fibroblast cell adaptation in basal media without growth factors as a function of culture time.

FIG. 5C depicts chicken fibroblast cell adaptation in serum free basal media supplemented with growth factors as a function of culture time. The growth factors comprise insulin-like, epidermal-like, and fibroblast-like growth factors.

FIG. 6A depicts the adaption of C1F chicken cells in media with decreasing concentrations of FBS in the presence of ITSEEF as defined herein, as a function of culture time. FIG. 6B depicts chicken fibroblast cell adaptation to serum-free media indicating the population doubling time as a function of passage number. FIG. 6C depicts cell viability as a function of time for the cultures shown in FIGS. 6A and 6B.

FIG. 7A shows the storage modulus of wet chicken cell paste (duplicate), wet bovine cell paste (duplicate) and a 7% mung bean protein isolate (duplicate) solution as the temperature is increased from 30° C. to 95° C. FIG. 7B shows the storage modulus of the wet chicken cell paste, wet bovine cell paste and 7% mung bean protein isolate in response to increasing oscillation strain.

FIG. 8 is a photograph of an extrudate that contains 65% cultivated chicken cells. The extrudate has a fibrous structure very similar to the fibrous structure of a chicken breast from a chicken.

FIG. 9 is graph showing the hardness of conventional chicken breast, plant protein based extrudate that does not contain chicken cells and an extrudate that contains 65% cultivated chicken cells.

### DETAILED DESCRIPTION

The following description is presented to enable one of ordinary skill in the art to make and use the disclosed subject matter and to incorporate it in the context of applications. Various modifications, as well as a variety of uses in different applications, will be readily apparent to those skilled in the art, and the general principles defined herein may be applied to a wide range of embodiments. Thus, the present disclosure is not intended to be limited to the embodiments presented but is to be accorded the widest scope consistent with the principles and novel features disclosed herein.

### Definitions

As used herein, “adventitious” refers to one or more contaminants such as, but not limited to: viruses, bacteria, Mycoplasma, and fungi.

As used herein, “basal medium” refers to a non-supplemented medium which promotes the growth of many types of microorganisms and/or cells which do not require any special nutrient supplements.

As used herein, the term “batch culture” refers to a closed culture system with nutrient, temperature, pressure, aeration, and other environmental conditions to optimize growth. Because nutrients are not added, nor waste products removed during incubation, batch cultures can complete a finite number of life cycles before nutrients are depleted and growth stops.

As used herein, “cell paste” or “wet cell paste” refers to a paste of cells harvested from a cell culture in which a desired amount of water has been removed from the harvested cell culture medium. It is within the ambit of skilled practitioners to remove water from the cell culture medium to produce cell paste comprising cultivated cells. According to the United States Department of Agriculture, the naturally occurring moisture content of animal meats including poultry, is about 75% water. The skilled worker can remove moisture from the harvested cultured cell media to produce cell paste by centrifugation, lyophilization, heating or any other well-known techniques to remove water. In some embodiments, the cell paste provided herein comprises a significant amount of water. Cell paste or wet cell paste” as used herein comprises about 1%-99%, 5%-99%, 10%-99%, 20%-99%, 25%-99%, 25%-99%, 25%-99%, 25%-95%, 25%-90% water 25%-85% water, 25%-80% water, 25%-75% water, 25%-70% water, 25%-65% water, 25%-60% water, 25%-55% water, 25%-50% water, 30%-90% water, 30%-85% water, 30%-80% water, 30%-75% water, 30%-70% water, 30%-65% water, 30%-60% water, 30%-55% water, 30%-50% water, 35%-90% water, 35%-85% water, 35%-80% water, 35%-75% water, 35%-70% water, 35%-65% water, 35%-60% water, 35%-55% water, 35%-50% water, 40%-90% water, 40%-85% water, 40%-80% water, 40%-75% water, 40%-70% water, 40%-65% water, 40%-60% water, 40%-60% water, 40%-55% water, 40%-50% water, 45%-90% water, 45%-85% water, 45%-80% water, 45%-75% water, 45%-70% water, 45%-75% water, 45%-70% water, 45%-65% water, 45%-60% water, 45%-55% water, 45%-50% water, 50%-90% water, 50%-85% water, 50%-80% water, 50%-75% water, 50%-70% water, 50%-65% water, 50%-60% water, 50%-55% water, 85%-99% water, 85%-95% water, 90%-99% water, 90%-95% water, 95%-99% water, or 95%-98% water. Cell paste or wet cell paste is another term for cultured meat. A cell paste, if dried sufficiently, can appear as a powder.

As used herein, the term “cultivated animal cell” is a cell originally obtained from an animal or a part of an animal that is cultivated or propagated in a growth medium.

As used herein, the term “dough” refers to a mixture of water and plant protein and optionally, the at least one other ingredient and, also optionally, a peptide cross-linking agent.

As used herein, the term “dough/cell admixture” refers to a combination of water, plant protein and cultivated animal cells, and optionally, the at least one other ingredient also optionally a peptide cross-linking agent.

As used herein, the term “edible food product” refers to a food product safe for human consumption. For example, this includes, but is not limited to a food product that is generally recognized as safe per a government or regulatory body (such as the United States Food and Drug Administration). In certain embodiments, the food product is considered safe to consume by a person of skill. Any edible food product suitable for a human consumption should also be suitable for consumption by another animal and such an embodiment is intended to be within the scope herein.

As used herein, the term “enzyme” or “enzymatically” refers to biological catalysts. Enzymes accelerate, or catalyze, chemical reactions. Enzymes increase the rate of reaction by lowering the activation energy.

As used herein, the term “expression” is the process by which information from a gene is used in the synthesis of a functional gene product.

As used herein, the term “extrudate” or “extruded product” refers to a product comprising cultivated animal cells, plant protein, at least one other ingredient, and optionally a peptide cross-linking enzyme, the extrudate is prepared by use of an extrusion machine. The extrudate can optionally comprise a peptide cross-linking enzyme.

As used herein, the term “fed-batch culture” refers to an operational technique where one or more nutrients, such as substrates, are fed to a bioreactor in continuous or periodic mode during cultivation and in which product(s) remain in the bioreactor until the end of a run. An alternative description is that of a culture in which a base medium supports initial cell culture and a feed medium is added to prevent nutrient depletion. In a fed-batch culture one can control concentration of fed-substrate in the culture liquid at desired levels to support continuous growth.

As used herein, the term “fiber” is carbohydrate polymer obtained from plants that cannot be completely broken down by the human digestive system when consumed. Soluble fiber is soluble in water and insoluble fiber does not dissolve in water.

As used herein, the term “fibrous” or “fibrous structure” is used to refer to extrudates or other products in which macromolecules such as protein fibers and/or cultivated cells are substantially aligned in one direction. A majority of the plant protein fibers and/or plant protein fibers that are covalently linked to cultivated animal cells that are present in the extrudate align with each other at an angle of about 750 angle or less, 650 angle or less, 600 angle or less, 550 angle or less, 500 angle or less, 450 angle or less, or 400 angle or less, when viewed from a top-down view.

As used herein, “fibroblasts” refers to mesenchymal-derived cells that are responsible for the extracellular matrix, epithelial differentiation, and regulation of inflammation and wound healing. In addition, fibroblasts are also responsible for the secretion of growth factors and work as scaffolds for other cell types. Fibroblasts are one cell type found in conventional meat.

As used herein, the term “gelation,” “gelling” or the like is a property of a material, for example, wet cell paste or protein to form a gel, wherein the material after gelation (gelled material) demonstrates a viscoelastic or elastic solid behavior compared to the ungelled material. The material can convert from an ungelled state to a gelled state by exposure to heat, addition of cross-linking agent, agitation or incubation under desired conditions. The gelation of materials, such as isolated proteins, is a commonly observed phenomenon. For example, eggs when exposed to heat, acids such as vinegar or salt will gel (curdle). Scrambled eggs are a gelled food product. Similarly, aqueous solutions of collagen will gel and form gelatin at particular temperatures and concentrations.

As used herein, a “gene product” is the biochemical material, either RNA or protein, resulting from expression of a gene.

As used herein, “growth medium” refers to a medium or culture medium that supports the growth of microorganisms or cells or small plants. A growth medium may be, without limitation, solid or liquid or semi-solid. Growth medium shall also be synonymous with “growth media.”

As used herein, the term “humectant” is a substance that is hygroscopic. Examples of humectants include unmodified starch, modified starch, and polyols. Xanthan gum, guar gum, alginate, carrageenan, glycerol, propylene glycol and polydextrose are commonly used humectants in food products.

As used herein, “in vitro” refers to a process performed or taking place in a test tube, culture dish, bioreactor, or elsewhere outside a living organism. In the body of this disclosure, a product may also be referred to as an in vitro product, in which case in vitro shall be an adjective and the meaning shall be that the product has been produced with a method or process that is outside a living organism.

As used herein, the term “lipid” or “fat” refers to compounds that are soluble in nonpolar solvents. Examples of lipids include triacylglycerols, diacylglycerols, monoacylglycerols, free fatty acids, and sterols.

As used herein “peptide cross-linking enzyme” or “cross-linking enzyme is an enzyme that catalyzes the formation of covalent bonds between one or more polypeptides. Transglutaminase is an example of a peptide cross-liking enzyme.

As used herein, “proliferation” refers to a process that results in an increase in the number of cells. It is characterized by a balance between cell division and cell loss through cell death or differentiation.

As used herein, “primary avian fibroblast cells” refers to cells from a parental animal that maintain growth in a suitable growth medium, for instance under controlled environmental conditions. Cells in primary culture have the same karyotype (number and appearance of chromosomes in the nucleus of a eukaryotic cell) as those cells in the original tissue.

As used herein “protein concentrate” is a collection of one or more different polypeptides obtained from a plant source or animal source. The percent protein by dry weight of a protein concentrate is greater than 25% protein by dry weight.

As used herein “protein isolate” is a collection of one or more different polypeptides obtained from a plant source or an animal source. The percent protein by dry weight of a protein isolate is greater than 50% protein by dry weight.

As used herein, “seasoning” refers to one or more herbs and spices in both solid and liquid form.

As used herein, “secondary avian fibroblast cells” refers to primary cells that have undergone a genetic transformation and become immortalized allowing for indefinite proliferation.

As used herein, the terms “storage modulus” is a measure of the stiffness of a viscoelastic material. The storage modulus is the measured elastic response of the material in response to a perturbation imparted to the viscoelastic material. The storage modulus is a unit of force, typically measure in Pascal.

As used herein, “substantially pure” refers to cells that are at least 80% cells by dry weight. Substantially pure cells are between 80%-85% cells by dry weight, between 85%-90% cells by dry weight, between 90%-92% cells by dry weight, between 92%-94% cells by dry weight, between 94%-96% cells by dry weight, between 96%-98% cells by dry weight, between 98%-99% cells by dry weight.

As used herein, “suspension culture” refers to a type of culture in which single cells or small aggregates of cells multiply while suspended in agitated liquid medium. It also refers to a cell culture or a cell suspension culture.

As used herein, “transglutaminase” or “TG” refers to an enzyme (R-glutamyl-peptide amine glutamyl transferase) that catalyzes the formation of a peptide (amide) bond between γ-carboxyamide groups and various primary amines, classified as EC 2.3.2.13. Transglutaminases catalyze the formation of covalent bonds between polypeptides, thereby cross-linked polypeptides. Cross-linking enzymes such as transglutaminase are used in the food industry to improve texture of some food products such as dairy, meat and cereal products. It can be isolated from a bacterial source, a fungus, a mold, a fish, a mammal, or a plant.

As used herein, and unless otherwise indicated, percentage (%) refers to total % by weight typically on a dry weight basis unless otherwise indicated.

The term “about” indicates and encompasses an indicated value and a range above and below that value. In certain embodiments, the term “about” indicates the designated value±10%, ±5%, or ±1%. In certain embodiments, the term “about” indicates the designated value±one standard deviation of that value.

In this disclosure, methods are presented for culturing avian derived cells in vitro. The methods herein provide methods to proliferate, recover, and monitor the purity of cell cultures. The cells can be used, for example, in one or more food products.

The disclosure herein sets forth embodiments for avian food products compositions comprising avian derived cells grown in vitro. In some embodiments, the compositions comprise plant protein, cell paste, fat, water, and a peptide cross-linking enzyme.

The disclosure herein sets forth embodiments for methods to prepare an avian food product made from avian derived cells grown in vitro. The avian food product is an edible food product.

### Cells

In some embodiments, the cultivated animal cells are avian cells, bovine cells, porcine cells, or seafood cells. Seafood cells include cells derived from animal cells that live in oceans, rivers lakes and riparian habitats. Seafood cells include but are not limited to fish cells, mollusk cells, crustacean cells and the cells of other organisms that are comestible. In some embodiments, the avian cells are selected from, but not limited to: chicken, pheasant, goose, swan, squab, pigeon, turkey, and duck. In some embodiments, the cells comprise primary avian fibroblast cells. In some embodiments, the cells comprise secondary avian fibroblast cells. In some embodiments, the cultivated animal cells are cells of the genus Gallus, Meleagris, Anas, Bos or Sus.

In some embodiments, the cells are UMNSAH/DF1 (C1F) cells. In certain embodiments, the cells are a commercially available chicken cell line deposited at American Type Culture Collection (ATCC, Manassas, Va., USA) on Oct. 11, 1996. In some embodiments, the cells used are derived from ATCC deposit number CRL12203.

In some embodiments, the avian cell lines have a spontaneously immortalized fibroblast phenotype. In some embodiments, the avian cell lines have high proliferation rates. In certain embodiments, the cells have both an immortalized fibroblast phenotype and high proliferation rates.

In some embodiments, the cells are not recombinant or engineered in any way (i.e., non-GMO). In some embodiments, the cells have not been exposed to any viruses and/or viral DNA. In certain embodiments, the cells are both not recombinant or have not been exposed to any viruses and/or viral DNA and/or RNA.

In some embodiment, a cell paste of the cultivated cells is prepared such that the concentration of the cells of the cell paste is between 1×10⁹ to 100×10⁹ cells per ml. Wet cell paste can be prepared by harvesting the cells from the bioreactor and dewatering the harvested culture medium to concentrate the cells. Alternatively, the culture medium in the bioreactor can be harvested at a desired cell density, in which case the dewatering step can be eliminated or the culture medium can be dewatered minimally to achieve the desired cell density of the wet cell paste. After completion of the cultivation of the cells, the culture medium containing the cultivated cells is harvested and water is removed to increase the concentration of the cells in the culture medium to produce cell paste or wet cell paste. For example, after harvesting 1000 L of culture medium, 999 L of the water (and other media components) present in the culture medium is removed to produce 1 L of cell paste, resulting in a 1,000 fold increase in the cell concentration. A harvested culture medium that contains 1×10⁶ cells per ml will produce cell paste that is 1×10⁹ cells per ml. Similarly, a harvested culture medium that contains 50×10⁶ cells per ml can be concentrated to produce cell paste that is 50×10⁹ cells per ml.

In some embodiments, the cell density of the wet cell paste is between 1×10⁹ to 100×10⁹ cells per ml, between 1×10⁹ to 90×10⁹ cells per ml, between 1×10⁹ to 80×10⁹ cells per ml, between 1×10⁹ to 70×10⁹ cells per ml is between 1×10⁹ to 60×10⁹ cells per ml, between 1×10⁹ to 50×10⁹ cells per ml, between 1×10⁹ to 40×10⁹ cells per ml, between 1×10⁹ to 30×10⁹ cells per ml, between 1×10⁹ to 20×10⁹ cells per ml, between 1×10⁹ to 10×10⁹ cells per ml, between 10×10⁹ to 20×10⁹ cells per ml, between 20×10⁹ to 30×10⁹ cells per ml, between 30×10⁹ to 40×10⁹ cells per ml, between 50×10⁹ to 60×10⁹ cells per ml, between 70×10⁹ to 80×10⁹ cells per ml, between 80×10⁹ to 90×10⁹ cells per ml, or between 90×10⁹ to 100×10⁹ cells per ml

In some embodiments, the wet cell paste at a temperature of between 30° C. and 95° C., has a storage modulus (G′) is between 5 Pa and 300 Pa, between 5 Pa and 250 Pa, between 5 Pa and 200 Pa, between 5 Pa and 150 Pa, between 5 Pa and 100 Pa, between 5 Pa and 90 Pa, between 5 Pa and 80 Pa, between 5 Pa and 70 Pa, between 5 Pa and 60 Pa, between 5 Pa and 50 Pa, between 5 Pa and 40 Pa, between 5 Pa and 30 Pa, between 5 Pa and 25 Pa, between 5 Pa and 20 Pa, between 5 Pa and 15 Pa, or between 5 Pa and 10 Pa.

In some embodiments, the gelation temperature of the wet cell paste is between 30° and 95° C., between 35° and 95° C., between 40° and 95° C., between 45° and 95° C., between 50° and 95° C., between 50° and 85° C., between 50° and 75° C., between 50° and 70° C., between 50° and 65° C., or between 50° and 60° C.

### Culture Media and Growth

In some embodiments, proliferation occurs in suspension or adherent conditions, with or without feeder-cells and/or in serum-containing or serum-free media conditions. In some embodiments, media for proliferation contains one or more of amino acids, peptides, proteins, carbohydrates, essential metals, minerals, vitamins, buffering agents, anti-microbial agents, growth factors, and/or additional components.

In some embodiments, proliferation is measured by any method known to one skilled in the art. In some embodiments, proliferation is measured through direct cell counts. In certain embodiments, proliferation is measured by a haemocytometer. In some embodiments, proliferation is measured by automated cell imaging. In certain embodiments, proliferation is measured by a Coulter counter.

In some embodiments, proliferation is measured by using viability stains. In certain embodiments, the stains used comprise trypan blue.

In some embodiments, proliferation is measured by the total DNA. In some embodiments, proliferation is measured by BrdU labelling. In some embodiments, proliferation is measured by metabolic measurements. In certain embodiments, proliferation is measured by using tetrazolium salts. In certain embodiments, proliferation is measured by ATP-coupled luminescence.

In some embodiments, the culture media is basal media. In some embodiments, the basal media is DMEM, DMEM/F12, MEM, HAMS's F10, HAM's F12, IMDM, McCoy's Media and RPMI.

In some embodiments, the basal media comprises amino acids. In some embodiments, the basal media comprises biotin. In some embodiments, the basal media comprises choline chloride. In some embodiments, the basal media comprises D-calcium pantothenate. In some embodiments, the basal media comprises folic acid. In some of embodiments, the basal media comprises niacinamide. In some embodiments, the basal media comprises pyridoxine hydrochloride. In some embodiments, the basal media comprises riboflavin. In some embodiments, thiamine hydrochloride is part of the basal media (DMEM/F12). In some embodiments, the basal media comprises vitamin B12 (also known as cyanocobalamin). In some embodiments, the basal media comprises i-inositol (myo-inositol). In some embodiments, the basal media comprises calcium chloride. In some embodiments, the basal media comprises cupric sulfate. In some embodiments, the basal media comprises ferric nitrate. In some embodiments, the basal media comprises magnesium chloride. In some embodiments, the basal media comprises magnesium sulfate. In some embodiments, the basal media comprises potassium chloride. In some embodiments, the basal media comprises sodium bicarbonate. In some embodiments, the basal media comprises sodium chloride. In some embodiments, the basal media comprises sodium phosphate dibasic. In some embodiments, the basal media comprises sodium phosphate monobasic. In some embodiments, the basal media comprises zinc sulfate. In some embodiments, the growth medium comprises sugars. In some embodiments, the sugars include but are not limited to D-glucose, galactose, fructose, mannose, or any combination thereof. In an embodiment, the sugars includes both D-glucose and mannose. In embodiments where glucose and mannose are both used in the growth medium to cultivate cells, the amount of glucose in the growth medium (cultivation media) is between 0.1-10 g/L, 0.1-9 g/L, 0.1-8 g/L, 0.1-7 g/L, 0.1-6 g/L, 0.1-5 g/L, 0.1-4 g/L, 0.1-3 g/L, 0.1-2 g/L, 0.1-1 g/L, 0.5-10 g/L, 0.5-9 g/L, 0.5-8 g/L, 0.5-7 g/L, 0.5-6 g/L, 0.5-5 g/L, 0.5-4 g/L, 0.5-3 g/L, 0.5-2 g/L, 0.5-1 g/L, 1-10 g/L, 1-9 g/L, 1-8 g/L, 1-9 g/L, 1-8 g/L, 1-7 g/L, 1-6 g/L, 1-5 g/L, 1-4 g/L, 1-3 g/L, 1-2 g/L, 2-10 g/L, 2-9 g/L, 2-8 g/L, 2-9 g/L, 2-8 g/L, 2-7 g/L, 2-6 g/L, 2-5 g/L, 2-4 g/L, 2-3 g/L, 3-10 g/L, 3-9 g/L, 3-8 g/L, 3-9 g/L, 3-8 g/L, 3-7 g/L, 3-6 g/L, 3-5 g/L, 3-4 g/L, 4-10 g/L, 4-9 g/L, 4-8 g/L, 4-9 g/L, 4-8 g/L, 4-7 g/L, 4-6 g/L, 4-5 g/L, 5-10 g/L, 5-9 g/L, 5-8 g/L, 5-9 g/L, 5-8 g/L, 5-7 g/L, or 5-6 g/L, and the amount of mannose in the growth media is between 0.1-10 g/L, 0.1-9 g/L, 0.1-8 g/L, 0.1-7 g/L, 0.1-6 g/L, 0.1-5 g/L, 0.1-4 g/L, 0.1-3 g/L, 0.1-2 g/L, 0.1-1 g/L, 0.5-10 g/L, 0.5-9 g/L, 0.5-8 g/L, 0.5-7 g/L, 0.5-6 g/L, 0.5-5 g/L, 0.5-4 g/L, 0.5-3 g/L, 0.5-2 g/L, 0.5-1 g/L, 1-10 g/L, 1-9 g/L, 1-8 g/L, 1-9 g/L, 1-8 g/L, 1-7 g/L, 1-6 g/L, 1-5 g/L, 1-4 g/L, 1-3 g/L, 1-2 g/L, 2-10 g/L, 2-9 g/L, 2-8 g/L, 2-9 g/L, 2-8 g/L, 2-7 g/L, 2-6 g/L, 2-5 g/L, 2-4 g/L, 2-3 g/L, 3-10 g/L, 3-9 g/L, 3-8 g/L, 3-9 g/L, 3-8 g/L, 3-7 g/L, 3-6 g/L, 3-5 g/L, 3-4 g/L, 4-10 g/L, 4-9 g/L, 4-8 g/L, 4-9 g/L, 4-8 g/L, 4-7 g/L, 4-6 g/L, 4-5 g/L, 5-10 g/L, 5-9 g/L, 5-8 g/L, 5-9 g/L, 5-8 g/L, 5-7 g/L, or 5-6 g/L. The skilled worker will understand that combinations of these amounts of glucose and mannose can be used, for example, between 2-5 grams of glucose and 1-4 grams of mannose.

In some embodiments, the basal media comprises linoleic acid. In some embodiments, the basal media comprises lipoic acid. In some embodiments, the basal media comprises putrescine-2HCl. In some embodiments, the basal media comprises 1,4 butanediamine. In some embodiments, the basal media comprises Pluronic F-68. In some embodiments, the basal media comprises fetal bovine serum. In certain embodiments, the basal media comprises each ingredient in this paragraph. In certain embodiments, the basal media is DMEM/F12.

In some embodiments, the growth medium comprises serum. In some embodiments, the serum is selected from bovine calf serum, chicken serum, and any combination thereof.

In some embodiments, the growth medium comprises at least 10% fetal bovine serum. In certain embodiments, the population of avian fibroblast cells are grown in a medium with at least 10% fetal bovine serum, followed by a reduction to less than 2% fetal bovine serum before recovering the cells.

In another embodiment, the culture media contains no serum including fetal bovine serum, fetal calf serum, or any animal derived serum.

In certain embodiments, the fetal bovine serum is reduced to less than or equal to 1.9% fetal bovine serum before recovering the cells. In certain embodiments, the fetal bovine serum is reduced to less than or equal to 1.7% fetal bovine serum before recovering the cells. In certain embodiments, the fetal bovine serum is reduced to less than or equal to 1.5% fetal bovine serum before recovering the cells. In certain embodiments, the fetal bovine serum is reduced to less than or equal to 1.3% fetal bovine serum before recovering the cells. In certain embodiments, the fetal bovine serum is reduced to less than or equal to 1.1% fetal bovine serum before recovering the cells. In certain embodiments, the fetal bovine serum is reduced to less than or equal to 0.9% fetal bovine serum before recovering the cells. In certain embodiments, the fetal bovine serum is reduced to less than or equal to 0.7% fetal bovine serum before recovering the cells. In certain embodiments, the fetal bovine serum is reduced to less than or equal to 0.5% fetal bovine serum before recovering the cells. In certain embodiments, the fetal bovine serum is reduced to less than or equal to 0.3% fetal bovine serum before recovering the cells. In certain embodiments, the fetal bovine serum is reduced to less than or equal to 0.1% fetal bovine serum before recovering the cells. In certain embodiments, the fetal bovine serum is reduced to less than or equal to 0.05% fetal bovine serum before recovering the cells. In certain embodiments, the fetal bovine serum is reduced to about 0% fetal bovine serum before recovering the cells.

In some embodiments, the basal media is DMEM/F12 and is in a ratio of 3:1; 2:1; or 1:1. In certain embodiments, the basal media is DMEM/F12 and in a ratio of about 3:1. In certain embodiments, the basal media is DMEM/F12 and in a ratio of about 2:1. In certain embodiments, the basal media is DMEM/F12 and in a ratio of about 1:1.

In some embodiments, the growth media is modified in order to optimize the expression of at least one gene from a cell signaling pathway selected from the group consisting of proteasome, steroid biosynthesis, amino acid degradation, amino acid biosynthesis, drug metabolism, focal adhesion, cell cycle, MAPK signaling, glutathione metabolism, TGF-beta, phagosome, terpenoid biosynthesis, DNA replication, glycolysis, gluconeogenesis, protein export, butanoate metabolism, and synthesis and degradation of ketone bodies.

In some embodiments, the steps of producing avian fibroblast are monitored for gene expression of one or more cell signaling pathways. In certain embodiments, the growth media is adjusted at each stage of cell production in accordance with data obtained from the monitoring of gene expression.

In some embodiments, the avian fibroblast cells are induced to accumulate lipids by adding or removing one or more compounds to or from the growth media in quantities sufficient to induce the accumulation of one or more lipids.

In some embodiments, one or more of the maintenance, proliferation, differentiation, lipid accumulation, lipid content, proneness to purification and/or harvest efficiency, growth rates, cell densities, cell weight, resistance to contamination, avian fibroblast-specific gene expression and/or protein secretion, shear sensitivity, flavor, texture, color, odor, aroma, gustatory quality, nutritional quality, minimized growth-inhibitory byproduct secretion, and/or minimized media requirements, of avian fibroblast cells, in any culture conditions, are improved by one or more of growth factors, proteins, peptides, fatty acids, elements, small molecules, plant hydrosylates, directed evolution, genetic engineering, media composition, bioreactor design, and/or scaffold design. In certain embodiments, the fatty acids comprise stearidonic acid (SDA). In certain embodiments, the fatty acids comprise linoleic acid. In certain embodiments, the growth factor comprises insulin or insulin like growth factor. In certain embodiments, the growth factor comprises fibroblast growth factor or the like. In certain embodiments, the growth factor comprises epidermal growth factor or the like. In certain embodiments, the protein comprises transferrin. In certain embodiments, the element comprises selenium. In certain embodiments, a small molecule comprises ethanolamine. The amount of ethanolamine used in the cultivations is between 0.05-10 mg/L, 0.05-10 mg/L, 0.1-10 mg/L, 0.1-9.5 mg/L, 0.1-9 mg/L, 0.1-8.5 mg/L, 0.1-8.0 mg/L, 0.1-7.5 mg/L, 0.1-7.0 mg/L, 0.1-6.5 mg/L, 0.1-6.0 mg/L, 0.1-5.5 mg/L, 0.1-5.0 mg/L, 0.1-4.5 mg/L, 0.1-4.0 mg/L, 0.1-3.5 mg/L, 0.1-3.0 mg/L, 0.1-2.5 mg/L, 0.1-2.0 mg/L, 0.1-1.5 mg/L, and 0.1-1.0 mg/L.

In certain embodiments, the media can be supplemented with plant hydrolysates. In certain embodiments, the hydrolysates comprise yeast extract, wheat peptone, rice peptone, phytone peptone, yeastolate, pea peptone, soy peptone, pea peptone, potato peptone, mung bean protein hydrolysate, or sheftone. The amount of hydrolysate used in the cultivations is between 0.1 g/L to 5 g/L, between 0.1 g/L to 4.5 g/L, between 0.1 g/L to 4 g/L, between 0.1 g/L to 3.5 g/L, between 0.1 g/L to 3 g/L, between 0.1 g/L to 2.5 g/L, between 0.1 g/L to 2 g/L, between 0.1 g/L to 1.5 g/L, between 0.1 g/L to 1 g/L, or between 0.1 g/L to 0.5 g/L.

In some embodiments, a small molecule comprises lactate dehydrogenase inhibitors. As described in the Examples below, lactate dehydrogenase inhibitors inhibit the formation of lactate. The production of lactate by avian cells inhibit the growth of the cells. Exemplary lactate dehydrogenase inhibitors are selected from the group consisting of oxamate, galloflavin, gossypol, quinoline 3-sulfonamides, N-hydroxyindole-based inhibitors, and FX11. In some embodiments, the amount of lactate dehydrogenase inhibitor in the fermentation medium is between 1-500 mM, 1-400 mM, 1-300 mM, 1-250 mM, between 1-200 mM, 1-175 mM, 1-150 mM, 1-100 mM, 1-50 mM, 1-25 mM, 25-500 mM, 25-400 mM, 25-300 mM, 25-250 mM, 25-200 mM, 25-175 mM, 25-125M, 25-100 mM, 25-75 mM, 25-50 mM, 50-500 mM, 50-400 mM, 50-300 mM, 50-250 mM, 50-200 mM, 50-175 mM, 50-150 mM, 50-125 mM, 50-100 mM, 50-75 mM, 75-500 mM, 75-400 mM, 75-300 mM, 75-250 mM, 75-200 mM, 75-175 mM, 75-150 mM, 75-125 mM, 75-100 mM, 100-500 mM, 100-400 mM, 100-300 mM, 100-250 mM, 100-200 mM, 100-150 mM, 100-125 mM, and 100-500 mM.

In some embodiments, the cultivated animal cells are grown in a suspension culture system. In some embodiments, the cultivated animal cells are grown in a batch, fed-batch, semi continuous (fill and draw) or perfusion culture system or some combination thereof. When grown in suspension culture, the suspension culture can be performed in a vessel (fermentation tank, bioreactor)) of a desired size. The vessel is a size that is suitable for growth of avian cells without unacceptable rupture of the cells. In some embodiments, the suspension culture system can be performed in vessel that is at least 25 liters (L), 50 L, 100 L, 200 L, 250 L, 350 L, 500 liters (L), 1000 L, 2,500 L, 5,000 L, 10,000 L, 25,000 L, 50,000 L, 100,000 L, 200,000 L, 250,000 L, or 500,000 L. For smaller suspension cultures, the cultivation of the cells can be performed in a flask that is least 125 mL, 250 mL, 500 mL, 1 L, 1.5 L, 2 L, 2.5 L, 3 L, 5 L, 10 L, or larger.

In some embodiments, the cell density of the suspension culture is between 0.25×10⁶ cells·ml, 0.5×10⁶ cells/ml and 1.0×10⁶ cells/ml, between 1.0×10⁶ cells/ml and 2.0×10⁶ cells/ml, between 2.0×10⁶ cells/ml and 3.0×10⁶ cells/ml, between 3.0×10⁶ cells/ml and 4.0×10⁶ cells/ml, between 4.0×10⁶ cells/ml and 5.0×10⁶ cells/ml, between 5.0×10⁶ cells/ml and 6.0×10⁶ cells/ml, between 6.0×10⁶ cells/ml and 7.0×10⁶ cells/ml, between 7.0×10⁶ cells/ml and 8.0×10⁶ cells/ml, between 8.0×10⁶ cells/ml and 9.0×10⁶ cells/ml, between 9.0×10⁶ cells/ml and 10×10⁶ cells/ml, between 10×10⁶ cells/ml and 15.0×10⁶ cells/ml, between 15×10⁶ cells/ml and 20×10⁶ cells/ml, between 20×10⁶ cells/ml and 25×10⁶ cells/ml, between 25×10⁶ cells/ml and 30×10⁶ cells/ml, between 30×10⁶ cells/ml and 35×10⁶ cells/ml, between 35×10⁶ cells/ml and 40×10⁶ cells/ml, between 40×10⁶ cells/ml and 45×10⁶ cells/ml, between 45×10⁶ cells/ml and 50×10⁶ cells/ml, between 50×10⁶ cells/ml and 55×10⁶ cells/ml, between 55×10⁶ cells/ml and 60×10⁶ cells/ml, between 60×10⁶ cells/ml and 65×10⁶ cells/ml, between 70×10⁶ cells/ml and 75×10⁶ cells/ml, between 75×10⁶ cells/ml and 80×10⁶ cells/ml, between 85×10⁶ cells/ml and 90×10⁶ cells/ml, between 90×10⁶ cells/ml and 95×10⁶ cells/ml, between 95×10⁶ cells/ml and 100×10⁶ cells/ml, between 100×10⁶ cells/ml and 125×10⁶ cells/ml, or between 125×10⁶ cells/ml and 150×10⁶ cells/ml.

In some embodiments, the cultivated animal cells are grown while embedded in scaffolds or attached to scaffolding materials. In some embodiments, the avian fibroblast cells are differentiated or proliferated in a bioreactor and/or on a scaffold. In some embodiments, the scaffold comprises at least one or more of a microcarrier, an organoid and/or vascularized culture, self-assembling co-culture, a monolayer, hydrogel scaffold, decellularized avian fibroblasts and/or an edible matrix. In some embodiments, the scaffold comprises at least one of plastic and/or glass or other material. In some embodiments, the scaffold comprises natural-based (biological) polymers chitin, alginate, chondroitin sulfate, carrageenan, gellan gum, hyaluronic acid, cellulose, collagen, gelatin, and/or elastin. In some embodiments, the scaffold comprises a protein or a polypeptide, or a modified protein or modified polypeptide. The unmodified protein or polypeptide or modified protein or polypeptide comprises proteins or polypeptides isolated from plants or other organisms. Exemplary plant protein isolates or plant protein concentrates comprise pulse protein, Vetch protein, grain protein, nut protein, macroalgal protein, microalgal protein, and other plant proteins. Pulse protein can be obtained from dry beans, lentils, mung beans, faba beans, dry peas, chickpeas, cowpeas, bambara beans, pigeon peas, lupins, vetches, adzuki, common beans, fenugreek, long beans, lima beans, runner beans, or tepary beans, soybeans, or mucuna beans. Vetch protein can be obtained from the genus Vicia. Grain protein can be obtained from wheat, rice, teff, oat, corn, barley, sorghum, rye, millet, triticale, amaranth, buckwheat, quinoa and other grains. Nut protein can be obtained from almond, cashew, pecan, peanut, walnut, macadamia, hazelnut, pistachio, brazil, chestnut, kola nut, sunflower seeds, pumpkin seeds, flax seeds, cacao, pine nut, ginkgo, and other nuts. Proteins obtained from animal source can also be used as scaffolds, including milk proteins, whey, casein, egg protein, and other animal proteins. In some embodiments, the self-assembling co-cultures comprise spheroids and/or aggregates. In some embodiments, the monolayer is with or without an extracellular matrix. In some embodiments, the hydrogel scaffolds comprise at least one of hyaluronic acid, alginate and/or polyethylene glycol. In some embodiments, the edible matrix comprises decellularized plant tissue. In some embodiments, the scaffolds used to cultivate animal cells is an extrudate of cultivated animal cells as described herein. In an embodiment, extrudates of the invention are first prepared and then used as a scaffold on which to cultivate animal cells.

In some embodiments, either primary or secondary avian fibroblast cells are modified or grown as in any of the preceding paragraphs.

### Recovery of Cells

The cultivated animal cells can be recovered by any technique known to those of skill. In some embodiments the avian fibroblast cells are separated from the growth media or are removed from a bioreactor or a scaffold. In certain embodiments, the avian fibroblast cells are separated by centrifugation, a mechanical/filter press, filtration, flocculation or coagulation or gravity settling or drying or some combination thereof. In certain embodiments, the filtration method comprises tangential flow filtration, vacuum filtration, rotary vacuum filtration and similar methods. In certain embodiments the drying can be accomplished by flash drying, bed drying, tray drying and/or fluidized bed drying and similar methods. In certain embodiments, the avian fibroblasts are separated enzymatically. In certain embodiments, the avian fibroblasts are separated mechanically.

### Cell Safety

In some embodiments, the population of cultivated animal is substantially pure.

In some embodiments, tests are administered at one or more steps of cell culturing to determine whether the cultivated animal cells are substantially pure.

In some embodiments, the cultivated animal cells are tested for the presence or absence of bacteria. In certain embodiments, the types of bacteria tested include, but are not limited to: Salmonella enteritidis, Staphylococcus aureus, Campylobacter jejunim, Listeria monocytogenes, Fecal streptococcus, Mycoplasma genus, Mycoplasma pulmonis, Coliforms, and Escherichia coli.

In some embodiments, components of the cell media, such as Fetal Bovine Serum, are tested for the presence or absence of viruses. In certain embodiments, the viruses include, but are not limited to: Bluetongue, Bovine Adenovirus, Bovine Parvovirus, Bovine Respiratory Syncytial Virus, Bovine Viral Diarrhea Virus, Rabies, Reovirus, Adeno-associated virus, BK virus, Epstein-Barr virus, Hepatitis A virus, Hepatitis B virus, Hepatitis C virus, Herpes Simplex 1, Herpes Simplex 2, Herpes virus type 6, Herpes virus type 7, Herpes virus type 8, HIV1, HIV-2, HPV-16, HPV 18, Human cytomegalovirus, Human Foamy virus, Human T-lymphotropic virus, John Cunningham virus, and Parvovirus B19.

In some embodiments, the tests are conducted for the presence or absence of yeast and/or molds.

In some embodiments, the tests are for metal concentrations by mass spectrometry, for example inductively coupled plasma mass spectrometry (ICP-MS). In certain embodiments, metals tested include, but are not limited to: arsenic, lead, mercury, cadmium, and chromium.

In some embodiments, the tests are for hormones produced in the culture. In certain embodiments, the hormones include, but are not limited: to 170-estradiol, testosterone, progesterone, zeranol, melengesterol acetate, trenbolone acetate, megestrol acetate, melengesterol acetate, chlormadinone acetate, dienestrol, diethylstilbestrol, hexestrol, taleranol, zearalanone, and zeranol.

In some embodiments, the tests are in keeping with the current good manufacturing process as detailed by the United States Food and Drug Administration.

### Phenotyping, Process Monitoring and Data Analysis

In some embodiments, the cells are monitored by any technique known to a person of skill in the art. In some embodiments, differentiation is measured and/or confirmed using transcriptional markers of differentiation after total RNA extraction using RT-qPCR and then comparing levels of transcribed genes of interest to reference, e.g. housekeeping, genes.

### Food Composition

In certain embodiments provided herein are extruded food compositions or food products comprising cultivated animal cells. In some embodiments, the cultivated animal cells are combined with other substances or ingredients to make a composition that is an extruded food product composition. In certain embodiments, the cultivated animal cells are used alone to an extruded food product. In certain embodiments, the extruded food product composition is a product that resembles: steak, nuggets, tenders, breasts, oysters, feet, wings, sausage, feed stock, or skin. In certain embodiments, the extruded product resembles a chicken product.

In some embodiments, the recovered cultivated animal cells are prepared into a composition with other ingredients. In certain embodiments, the composition comprises cell paste, plant protein, fat (lipid), and water.

In certain embodiments, the extruded food composition or food product has a wet cell paste content of at least 100%, 90%, 80%, 75%, 70%, 65%, 60%, 50%, 40%, 30%, 35%, 25%, 15%, 10%, 5% or 1% by weight. In certain embodiments, the extruded food composition or food product has a wet cell paste content by weight of between 10%-20%, 20%-30%, 30%-40%, 40%-50%, 60%-70%, 80%-90%, or 90%-100%. In certain embodiments, the extrudate or food product has a cultivated animal cell content by weight of between 10%-20%, 20%-30%, 30%-40%, 40%-50%, 60%-70%, 80%-90%, or 90%-100%. In certain embodiments, the composition comprises a plant protein content by weight of at least 75%, 70%, 60%, 50%, 40%, 30%, 25%, 20%, or 15% by weight. In certain embodiments, the extruded food product has a plant protein content by weight of between 10%-20%, 20%-30%, 30%-40%, 40%-50%, 60%-70%, 80%-90%, or 90%-95%. In certain embodiments, the extrudate comprises a fat content of at least 50%, 40%, 30%, 25%, 20%, 15%, 10%, 5%, or 1% by weight. In certain embodiments, the extruded food composition or food product has a fat content by weight of between 10%-20%, 20%-30%, 30%-40%, 40%-50%, 60%-70%, 80%-90%, or 90%-95%. In certain embodiments, the extrudate comprises a water content of at least 50%, 40%, 30%, 25%, 20%, 15%, 10% or 5% by weight. In certain embodiments, extrudate has a water content by weight of between 10%-20%, 20%-30%, 30%-40%, 40%-50%, 60%-70%, 80%-90%, or 90-95%. In certain embodiments, the extrudate comprises a wet cell paste content of between 2%-5%, 5%-10%, 10%-15%, 15%-20%, 20%-25%, 25%-30%, 30%-35%, 35%-40%, 40%-45%, 45%-50%, 50%-55%, 55%-60%, 65%-70%, 70%-75%, 75%-80%, 80%-85%, 85%-90%, or 90%-95%.

In some embodiments, the extrudate comprises at least one other ingredient. The at least one other ingredient is in an amount by weight of between 0.1% to 0.2%, between 0.2% to 0.3%, between 0.3% to 0.4%, between 0.4% to 0.5%, between 0.5% to 0.6%, between 0.6% to 0.7%, between 0.7% to 0.8%, between 0.8% to 0.9%, between 0.9% to 1%, between 1% to 2%, between 2% to 3%, between 3% to 4%, between 4% to 5%, between 5% to 7.5%, between 7.5% to 10%, between 10% to 12.5%, between 12.5% to 15%, between 15% to 20%, between 20% to 25%, between 25% to 30%, between 30% to 40%, between 40% to 50%, between 50% to 60%, or between 60% to 75%.

In some embodiments, the extrudate comprises a peptide cross-linking enzyme, for example, transglutaminase, content between 0.0001-0.025%.

In certain embodiments, the extrudate or food product comprises a dry cell weight content of at least of 1% by weight, 1% by weight, 5% by weight, 10% by weight, 15% by weight, 20% by weight, 25% by weight, 30% by weight, 35% by weight, 40% by weight, 45% by weight, 50% by weight, 55% by weight, 60% by weight, 65% by weight, 70% by weight, 75% by weight, 80% by weight, 85% by weight, 90% by weight, 95% by weight, or 99% by weight. In certain embodiments, the extrudate or food product comprises a dry cell weight content of between 2%-5%, 5%-10%, 10%-15%, 15%-20%, 20%-25%, 25%-30%, 30%-35%, 35%-40%, 40%-45%, 45%-50%, 50%-55%, 55%-60%, 65%-70%, 70%-75%, 75%-80%, 80%-85%, 85%-90%, or 90%-95%.

In certain embodiments, the extrudate or food product comprises a wet cell paste content of at least 1% by weight, at least 3% by weight, at least 5% by weight, at least 10% by weight, at least 15% by weight, at least 20% by weight, at least 25% by weight, at least 30% by weight, at least 35% by weight, at least 40% by weight, at least 45% by weight, at least 50% by weight, at least 55% by weight, at least 60% by weight, at least 65% by weight, at least 70% by weight, at least 75% by weight, at least 80% by weight, at least 85% by weight, at least 90% by weight, at least 95% by weight, or at least 99% by weight.

In certain embodiments, the extrudate or food product comprises a dry cell weight content of between 2%-5%, 5%-10%, 10%-15%, 15%-20%, 20%-25%, 25%-30%, 30%-35%, 35%-40%, 40%-45%, 45%-50%, 50%-55%, 55%-60%, 65%-70%, 70%-75%, 75%-80%, 80%-85%, 85%-90%, or 90%-95%.

In certain embodiments, the extrudate or food product comprises a pulse protein content of at least 2%, 5%, 10%, 15%, 20%, 25%, 30%, 35%, 40%, 45%, 50%, 55%, 60%, 65%, 70%, 75%, 80%, 85%, 90% or 95% by weight. In certain embodiments, the extrudate or food product comprises a pulse protein content of between 2%-5%, 5%-10%, 10%-15%, 15%-20%, 20%-25%, 25%-30%, 30%-35%, 35%-40%, 40%-45%, 45%-50%, 50%-55%, 55%-60%, 65%-70%, 70%-75%, 75%-80%, 80%-85%, 85%-90%, or 90%-95%, In some embodiments, the pulse protein is a mung bean protein.

In certain embodiments, the extrudate or food product comprises, a fat content of at least 1% by weight, a fat content of at least 2% by weight, a fat content of at least 5% by weight, a fat content of at least 7.5% by weight, or a fat content of at least 10% by weight, at least 15% by weight, at least 20% by weight, at least 25% by weight, at least 30% by weight, at least 35% by weight, at least 40% by weight, at least 45% by weight, at least 50% by weight, at least 55% by weight, at least 60% by weight, at least 70% by weight, at least 75% by weight, at least 80% by weight, at least 85% by weight, at least 90% by weight, or at least 95% by weight. In some embodiments, the extrudate or food product comprises a fat content of between 1%-5%, between 5%-10%, between 10%-15%, between 15%-20%, between 20%-25%, between 25%-30%, between 30%-35%, between 35%-40%, between 45%-50%, between 50%-55%, between 55%-60%, between 60%-65%, between 65%-70%, between 70%-75%, between 75%-80%, between 80%-85%, between 85%-90%, or between 90%-95%.

In certain embodiments, the extrudate or food product comprises a water content of at least 5% by weight, at least 10% by weight, at least 15% by weight, at least 20% by weight, at least 25% by weight, at least 30% by weight, at least 35% by weight, at least 40% by weight, at least 45% by weight, at least 50% by weight, at least 55% by weight, at least 60% by weight, at least 65% by weight, at least 70% by weight, at least 75% by weight, at least 80% by weight, at least 85% by weight, at least 90% by weight, or at least 95% by weight.

In one embodiment, the extrudate, or food product comprises a wet cell paste content between 25-75% by weight, a mung bean protein content between 15-45% by weight, a fat content between 10-30% by weight, and a water content between 20-50% by weight.

In certain embodiments, the extrudate or food product comprises peptide cross-linking enzyme. Exemplary peptide cross-linking enzymes are selected from the group consisting of transglutaminase, sortase, subtilisin, tyrosinase, laccase, peroxidase, and lysyl oxidase. In certain embodiments, the composition comprises a cross-linking enzyme of between 0.0001%-0.025%, 0.0001%-0.020%, 0.0001%-0.0175%, 0.0001%-0.0150%, 0.0001%-0.0125%, 0.0001%-0.01%, 0.0001%-0.0075%, 0.0001%-0.005%, 0.0001%-0.0025%, 0.0001%-0.002%, 0.0001%-0.0015%, 0.0001%-0.001%, 0.0001%-0.00015% by weight. In certain embodiments, the food composition or food product comprises a transglutaminase content between 0.0001%-0.025%, 0.0001%-0.020%, 0.0001%-0.0175%, 0.0001%-0.0150%, 0.0001%-0.0125%, 0.0001%-0.01%, 0.0001%-0.0075%, 0.0001%-0.005%, 0.0001%-0.0025%, 0.0001%-0.002%, 0.0001%-0.0015%, 0.0001%-0.001%, 0.0001%-0.00015% by weight. Without being bound by theory, the peptide cross-linking enzyme is believed to cross-link the pulse or Vetch proteins and the peptide cross-linking enzyme is believed to cross-link the pulse or Vetch proteins to the avian cells.

In one embodiment, the extrudate or food product comprises 0.0001% to 0.0125% transglutaminase, and exhibits reduced or significantly reduced lipoxygenase activity or other enzymes which oxidize lipids, as expressed on a volumetric basis relative to cell paste without the transglutaminase. More preferably, the extrudate or food product is essentially free of lipoxygenase or enzymes that can oxidize lipids. In some embodiments, a 5%, 10%, 15%, 20%, 25%, 30%, 35%, 40%, 45%, 50%, 55%, 60%, 65%, 70%, 75%, or 80% reduction in oxidative enzymatic activity relative to a composition is observed. Lipoxygenases catalyze the oxidation of lipids that contribute to the formation of compounds that impart undesirable flavors to compositions.

In some embodiments, mung bean protein is replaced by plant-based protein comprising protein from garbanzo, fava beans, yellow pea, sweet brown rice, rye, golden lentil, chana dal, soybean, adzuki, sorghum, sprouted green lentil, du pung style lentil, and/or white lima bean.

In some embodiments, the addition of additional edible ingredients can be used to prepare the food composition of food product. Edible food ingredients comprise texture modifying ingredients such as humectants such as starches, modified starches, gums and other hydrocolloids. Other food ingredients comprise pH regulators, anti-caking agents, colors, emulsifiers, flavors, flavor enhancers, foaming agents, anti-foaming agents, humectants, sweeteners, and other edible ingredients. The addition of humectants causes the retention of moisture in the extrudate. In addition, while not being bound to theory, it is believed that the addition of humectants create more fibrous structures in the extrudate.

In certain embodiments, the methods and food composition or food product comprise an effective amount of an added preservative in combination with the food combination.

Preservatives prevent food spoilage from bacteria, molds, fungi, or yeast (antimicrobials); slow or prevent changes in color, flavor, or texture and delay rancidity (antioxidants); maintain freshness. In certain embodiments, the preservative is one or more of the following: ascorbic acid, citric acid, sodium benzoate, calcium propionate, sodium erythorbate, sodium nitrite, calcium sorbate, potassium sorbate, BHA, BHT, EDTA, tocopherols (Vitamin E) and antioxidants, which prevent fats and oils and the foods containing them from becoming rancid or developing an off-flavor.

The extruded food product has a texture similar to that of meat obtained from a farmed animal. The Warner-Bratzler shear force test measures the force required to cut through a piece of farm raised meat, cultured meat or extruded cultured meat. In other words, the Wamer-Bratzler score is a measure of the hardness of the meat. Wamer-Bratzler shear force testing equipment is commercially available from a number of manufacturers. The Warner-Bratzler score is a measure of force measure in Newtons or pounds. In some embodiments, the Wamer-Bratzler score of the extrudate or the food product is between 5 N to 300 N, between 5 N to 50 N, between 50 N to 75 N, between 75 N to 100 N, between 100 N to 125 N, between 125 N to 150 N, between 150 N to 175 N, between 175 N to 200 N, between 200 N to 225 N, between 225 N to 250 N, between 250 N to 275 N, and between 275 N to 300 N.

Another method to measure the hardness of a food product is to use a puncture test to measure the force required to puncture the food product to a predefined depth. There are commercially available texture analyzer that will measure the force needed to puncture the cultured meat product. An exemplary texture analyzer is the TA.XTplus Texture Analyser manufactured by Stable Micro Systems. In some embodiments, the force needed to puncture the extruded food product has a hardness as measured by a puncture test of between 1 N and 50 N, between 1 N and 45 N, between 1 N and 40 N, between 1 N and 35 N, between 1 N and 30 N, between 1 N and 25 N, between 1 N and 20 N, between 1 N and 15 N, between 1 N and 10 N, between 1 N and 9 N, between 1 N and 8 N, between 1 N and 7 N, between 1 N and 6 N, between 1 N and 5 N, between 1 N and 4 N, between 1 N and 3 N, between 1 N and 2 N, between 2 N and 15 N, between 2 N and 10 N, between 2 N and 9 N, between 2 N and 8N, between 2 N and 7 N, between 2 N and 6 N, between 2 N and 5 N, between 2 N and 4 N, or between 2 N and 3 N,

The extrudate in some embodiments is used as a substrate for 3D printing. In some embodiments, the 3D printed material is edible. 3D printing is a manufacturing process in which, starting with a substrate, various desired layers are added to the substrate in a sequential manner. The 3D printing process has been described as an “additive process.” In 3D printing of food stuffs, the process begins with an edible substrate and additional edible materials is deposited onto the substrate. In some embodiments, the extrudate as described herein is used as the substrate for 3D printing. In some embodiments, various cells such as fibroblasts, fat cells, muscle cells, and other cell types are deposited onto the extrudate. In addition, other ingredients such as but not limited to fats, salt, sugar, spices, flavorants, odorants, polymers such as polysaccharides and/or polypeptides are deposited onto the extrudate.

### Extrusion Process

In some embodiments, provided herein are methods of preparing extruded food products. The extruded food products are prepared in an extruder.

Extrusion cooking is a thermo-mechanical operation providing continuous mixing, kneading, and shaping. Four types of commonly used extruders include: single-screw wet extruders, single-screw dry extruders, single-screw interrupted-flight extruders, and twin-screw extruders. The twin-screw extruders can be used in wet extrusion methods or dry extrusion methods.

The extrusion process described herein is comprises: A dough or a dough/cell admixture is prepared and is placed into a hopper connected with either a volumetric feeder vs gravimetric feeder into the stationary barrel of the extruder. The dough or the dough/cell admixture is fed into the barrel and is conveyed by mechanical pressure through the passage between rotating screw(s) and the stationary barrel by the rotating screw(s). The stationary barrel can be heated, usually steam-heated. As the dough or the dough/cell admixture is conveyed along the barrel, one or more ports can be used for injection of liquid ingredients, including cultivated animal cells, oil, water, sugar solutions and/or the at least one other ingredient. As the dough or dough/cell admixture is conveyed to the end of the barrel, the extrudate exits the extrusion machine through a die, typically a small outlet. In some embodiments, the dough or the dough/cell admixture is heated. The dough or the dough/cell admixture can be heated, as the dough or the dough/cell admixture is conveyed through the barrel, by heating the stationary barrel or by injecting steam into the barrel.

Depending on the desired physical properties of the extrudate, a wet extrusion process or a dry extrusion process can be employed. While not being bound by theory, it is believed that a wet extrusion process produces extrudates with a more pronounced fibrous structure. It is believed that the laminar flow of the dough or the dough/cell admixture during conveyancing through the barrel, the protein molecules and/or the protein molecules covalently linked to cultivated animal cells undergo laminar flow and are realigned into fibrous strands, leading to a product that is similar to the texture of meat obtained from a farmed animal.

In a “wet extrusion” process, the moisture content of the dough or the dough/cell admixture is above 40%. The moisture content of the dough or the dough/cell admixture ins a wet extrusion process is 40%-45%, 45%-50%, 50%-55%, 55%-60%, 60%-65%, 65%-70%, 75%-80%, 80%-85%, 85%-90%, or 90%-95%.

In a “dry extrusion” process, the moisture content of the dough or the dough/cell admixture in a dry extrusion process is below 40%. The moisture content of the dough or the dough/cell admixture in the dry extrusion process is 5%-10%, 10%-15%, 15%-20%, 20%-25%, 25%-30%, or 35%-40%.

In some embodiments, dry ingredients are placed in the hopper, the dry ingredients comprise plant protein. In some embodiment, the dry ingredients comprise plant protein, the at least one other ingredient and/or dry peptide-cross linking agent to prepare a dry ingredient mixture. In other embodiments, the dry ingredients comprise plant and dried cultivated animal cells. The cultivated animal cells can be dried using conventional drying methods including freeze-drying or dried by applying heat and/or vacuum to the cultivated animal cells. The dry ingredient mixture is placed into the barrel of an extrusion at a desired loading rate. As the dry ingredient mixture is conveyed through the barrel, a dough or a dough/cell admixture is prepared by injecting steam or water into the barrel. The dough or the dough/cell admixture is conveyed through the barrel and is optionally heated. In some embodiments where the cultivated animal cells is not a dry ingredient, the cultivated animal cell is injected into the barrel to prepare the dough/cell admixture. In other embodiments. The dough/cell admixture is extruded through a die to prepare the extrudate.

The screw configuration of the extruder can be categorized in two general types: single screw and twin screw. In a single screw extruder, the screw design could have various configurations including decreasing pitch, increasing core, threaded barrel, conical barrel or alternating pin-type screw, or other screw design. In a twin screw extruder, there are two screws. The two screws can be arranged to rotate in a concurrent manner or in a countercurrent manner. In concurrent twin-screw designs, the two screws rotate in the same direction, that is, both screws rotate clockwise or counterclockwise. In concurrent twin-screw designs, the two screws counterrotate, that is, the one (first) screw rotates clockwise and the other (second) screw rotates counterclockwise. In both concurrent and concurrent designs, the two screws can intermesh or not intermesh (tangential). In non-intermeshing designs, the interaxial distance of the two screws are designed such that the flight path of one screw does not infiltrate the flight path of the second screw. In intermeshing twin-screw designs, the interaxial distance of the screws are designed so that the flight path of one screw infiltrates the flight path of the second screw. Depending on the depth of the infiltration, the screw configuration can be described as self-cleaning or partially self-cleaning. In addition, the handedness of the screw can be varied. One or both screws of the extruder can be right-handed or left-handed as viewed from a particular perspective, either downstream or upstream.

Modern extrusion machines often use modular screw designs that allows use of different screws having different configurations to quickly implement process changes. The screw configuration is designed in a way to optimize the drag speed, pressure speed and other parameters to achieve optimal mixing, shearing and/or heat transfer to obtain the desired laminated internal texture of cultured cell extrudates. The screw assembly in one embodiment, can be the following: 1) fully intermeshing counter-rotating twin screw design; 2) fully intermeshing co-rotating twin screw design; 3) non-intermeshing (tangential) counter-rotating twin screw design; 4) non-intermeshing (tangential) co-rotating twin screw design. Splined shafts are usually employed to hold screw sections of varying configuration: forward pitch, reverse screw, forward paddle and reverse paddle, kneading disks, compression disks.

The screw comprises one or more sections including the transport section and kneading section (sometimes called a melting zone), and may also comprise a metering section where the melted material is subjected to additional mixing before expulsion through the die. The radius of the screw depends on the size of extrusion unit. The radius of the screw can be as small as 8 mm for bench extrusion machines, or as large as 100 mm or larger for commercial production machines. The pitch of the screws can be of various sizes and angles. The pitch of the various portions of the screw usually have an angle of between 30°-90°. The pitch angle (helix angle) of the screw can be 30°-35°, 35°-40°, 40°-45°, 45°-50°, 50°-55°, 55°-60°, 60°-65°, 65°-70°, 70°-75°, 75°-80°, 80°-85°, or 85°-90°. Typically, the greater the angle, the stronger the shear force. The rotational frequency of the screw relative to the diameter of the screw is a common design parameter of the screw. For a screw that is 0.5D pitch, the screw completes one full rotation (360°) in 0.5 diameters. Common screws 0.25D, 0.5D, 0.75D, and 1.0D, and screws with other pitch parameters are commercially available and also can be customized by suppliers

The barrel design of extruders for counterrotating type of intermeshing twin-screw design, the barrel can either be a cylindrical shape or conical shape.

The pressure in the barrel can differ in the different zones of the screw. The pressures in the different zones can be as low as 1 bar up to 300 bar and beyond. For dry extrusion, the pressures inside the barrel are typically higher than in wet extrusion. The pressures in the barrels of the extrusion machines used to prepare extrudates described herein can be between 1-10 bar, 10-20 bar, 20-30 bar, 30-40 bar, 40-50 bar, 50-60 bar, 60-70 bar, 70-80 bar, 80-90 bar, 90-100 bar, 100-110 bar, 110-120 bar, 120-130 bar, 130-140 bar, 140-150 bar, 150-160 bar, 160-170 bar, 170-180 bar, 190-200 bar, 200-210 bar, 210-220 bar, 220-230 bar, 230-240 bar, 240-250 bar, 250-260 bar, 260-270 bar, 270-280 bar, 280-290 bar, or 290-300 bar.

The diameter, length, temperature, pressure, transit time and transit time through the barrel, screw configuration, screw speed, the feed rate from the hopper into the barrel, and other parameters as discussed herein are controlled.

During transit through the barrel and the die, the dough or the dough/cell admixture can be cooled or heated. Active cooling of certain portions of the die can be used to limit the expansion of the dough or the dough/cell admixture. Die nozzles with various diameters and length is used to control the pressure on the dough/cell admixture to control the expansion of the dough/cell admixture as it exits the die. In addition, the flow rate of the dough/cell admixture through die is also controlled. By limiting expansion, the extrudate has a more fibrous texture and replicates the mouthfeel and eating experience of meat from a farmed animal.

In some embodiments, provided herein are processes for making a food product that comprises combining pulse protein, cell paste and a phosphate into water and heating up the mixture in three steps. In certain embodiments, the processes comprise adding phosphate to water thereby conditioning the water to prepare conditioned water. In certain embodiments, pulse protein is added to the conditioned water in order to hydrate the pulse protein to prepare hydrated plant protein. In some embodiments, cell paste is added to the hydrated plant protein (conditioned water to which a plant protein has been added) to produce a cell protein mixture. In some embodiments, the plant protein is a pulse protein. In some embodiments, the pulse protein is a mung bean protein

In some embodiments, the phosphate is selected from the group consisting of disodium phosphate (DSP), sodium hexametaphosphate (SHMP), tetrasodium pyrophosphate (TSPP). In one particular embodiment, the phosphate added to the water is DSP. In some embodiments, the amount of DSP added to the water is at least or about 0.01%, 0.02%, 0.03%, 0.04%, 0.05%, 0.06%, 0.07%, 0.08%, 0.09%, 0.1%, 0.11%, 0.12%, 0.13%, 0.14%, 0.15%, or greater than 0.15%.

In some embodiments, the process comprises undergo three heating steps. In some embodiments, the first heating step comprises heating the cell and protein mixture to a temperature between 40-65° C., wherein seasoning is added. In some embodiments, the second step comprises maintaining the cell and protein mixture at temperature between 40-65° C. for at least 10 minutes, wherein a peptide cross-linking enzyme such as transglutaminase is added. In some embodiments, the third heating step comprises raising the temperature of the cell and protein mixture to a temperature between 60-85° C., where oil is added to the water. In some embodiments, the process comprises a fourth step of lowering the temperature to a temperature between 5-15° C. to prepare a pre-cooking product.

In some embodiments, the seasonings are added to the first step, second step, third step or the fourth step. In some embodiments the seasonings include but are not limited to salt, sugar, paprika, onion powder, garlic powder, black pepper, white pepper, and natural chicken flavor (Vegan).

In some embodiments, the oil (fat) added is to the first step, second step, third step or the fourth step to prepare the pre-cooking product. The oil is selected from the group comprising vegetable oil, peanut oil, canola oil, coconut oil, olive oil, corn oil, soybean oil, sunflower oil, margarine, vegetable shortening, animal oil, butter, tallow, lard, margarine, or an edible oil.

In some embodiments, the pre-cooking product can be consumed without additional preparation or cooking, or the pre-cooking product can be cooked further, using well-known cooking techniques.

In some embodiments, the processes comprise preparing the avian food product by placement into cooking molds. In some embodiments, the processes comprise applying a vacuum to the cooking molds effectively changing the density and texture of the avian food product.

In some embodiments, the avian food product is breaded.

In some embodiments, the avian food product is steamed, boiled, sautéed, fried, baked, grilled, broiled, microwaved, dehydrated, dried, cooked by sous vide, pressure cooked, or frozen or any combination thereof.

In some embodiments, the extrudate has a hardness as measured by a puncture test of between 1 N and 50 N, between 1 N and 45 N, between 1 N and 40 N, between 1 N and 35 N, between 1 N and 30 N, between 1 N and 25 N, between 1 N and 20 N, between 1 N and 15 N, between 1 N and 10 N, between 1 N and 9 N, between 1 N and 8 N, between 1 N and 7 N, between 1 N and 6 N, between 1 N and 5 N, between 1 N and 4 N, between 1 N and 3 N, between 1 N and 2 N, between 2 N and 15 N, between 2 N and 10 N, between 2 N and 9 N, between 2 N and 8N, between 2 N and 7 N, between 2 N and 6 N, between 2 N and 5 N, between 2 N and 4 N, or between 2 N and 3 N.

### EXAMPLES

### Example 1: Culturing Cells

Cells are daughter cell lines derived from the commercially available chicken cell line UMNSAH/DF1 (C1F is the Applicant's internal designation of the cells), deposited at American Type Culture Collection (ATCC, Manassas, Va., USA) on Oct. 11, 1996.

Media formulation is a basal media (DMEM/F12) comprising amino acids, vitamins, inorganic salts and other components supplemented with FBS or BCS (bovine calf serum).

### Creation of Master Working Cell Banks (MCWB)

A single vial of cells was retrieved from the C1F master cell bank (MCB) to establish C1F MWCB. Briefly, a C1F MCB cryovial was removed from the liquid nitrogen storage and immediately placed into a 37° C. water bath. The cell suspension was quickly thawed by gently swirling the vial. C1F cell suspension was gradually transferred into 15 mL conical tubes containing 10 mL of pre-warmed culture media in a laminar flow hood. The resultant diluted C1F cell suspension was centrifuged for 5 min at 300×g. The supernatant was aseptically aspirated without disturbing the cell pellet. C1F cells were gently resuspended in culture media and transferred into a 250 mL spin culture flask with a final working volume of 50 mL. Cell density and viability post-thawing were determined to monitor C1F health and for quality control of the established MCB.

C1F cells were cultured under agitation at 125 rpm for a total of 9 days and four steps of scale-up. First, the cells were cultured for 2 days at 37° C. in a humidified incubator with 5% C02. The culture was then centrifuged at 300×g for 5 min. Culture supernatant was decanted and C1F cell pellet was resuspended in fresh media and seeded in a final volume of 130 mL in a 500 mL shaking flask. Second, C1F cells were cultured under agitation at 125 rpm for additional 2 days at 37° C. in a humidified incubator with 5% C02. The culture was then centrifuged again at 300×g for 5 min; the cell pellet was resuspended in fresh media to a final volume of 340 mL of media in a 1 L shaking flask. Finally, after two days of culture, cell culture was collected and centrifuged at 300×g for 5 min; C1F cell pellet resuspended in fresh media for a final working volume of 880 mL in a 2 L shaking flask. C1F cells were cultured for 2 days under the same conditions, centrifuged at 300×g for 5 min; and the cell pellet was resuspended in fresh media to a final volume of 2.3 L in a 5 L shaking flask. C1F cell culture was placed for 1 additional day under agitation in a humidified incubator with 5% C02 and harvested for creation of MWCB.

C1F cells in the final expansion culture were collected and centrifuged at 300×g for 5 min. Cells were resuspended in lower volume of culture media and concentrated C1F cells were sampled and counted using semi-automated cell counting system (Vi-Cell). C1F cells went through another centrifugation cycle of 300×g for 5 min and were resuspended in cryopreservation media (with 10% DMSO) in a range of 20-25 million cell/mL. Cells were frozen in bar-coded cryovials at a rate of −1° C./min from 4° C. to −80° C. during a 16 to 24-hour period in isopropanol chambers. Cells were then transferred and stored in a vapor phase liquid nitrogen storage system (Taylor Wharton (<−175° C.)). Vial content and banked storage position were recorded in a controlled database.

CGMP chain of custody documentation (vial identity confirmation) was utilized to ensure the appropriate vial(s) are retrieved from the MWCB for cell bank release testing and cultured meat production.

### Example 2: Cultured Chicken Production

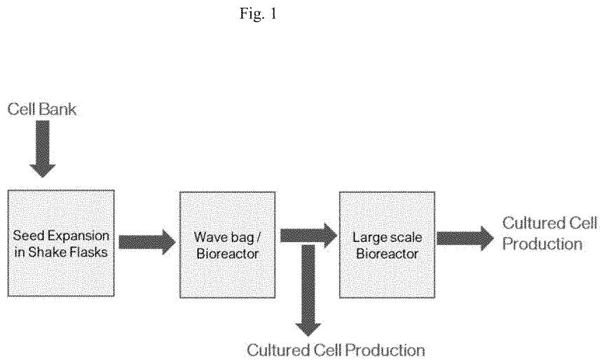

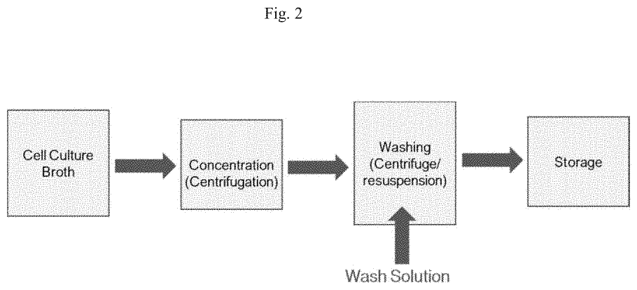

Single-use disposable systems were used for seed expansion and cell growth in the exemplary manufacturing process. The disposal systems with long contact time with the culture media include shake flasks, Wave Bags, media hold bags and stirred tank bioreactor bags for the large-scale 500 L bioreactors. FIG. 1 depicts a process diagram for cell culturing avian fibroblast cells. FIG. 2 depicts a process diagram for harvesting cells.

Seed expansion begins by thawing vials of cells from the MWCB and were cultured in a 500 mL shake flask with 100 mL of working volume. DMEM/F12 with 5% FBS is used in seed expansion. The culture is then split 1:3 to 1:6 and seeded into 1 L shaking flask with a working volume of 300 mL.

The scale-up culture of C1F cells in large shake flasks proceed with a 1:3 to 1:6 split ratio to 900 mL in a 3 L flask followed by 900 mL culture split to 2.7 L in a 5 L flask. Finally, the 2.7 L culture in 5 L flasks is further split in to three 2.7 L flasks using 1:3 split ratios for the transfer into the Wave Bag.

### Cell Culture in Wave Bag

Culture from the three 5 L shake flask (2.7 L culture) was used to inoculate a Wave Bag (total volume of 50 L with a maximum working volume of 25 L) under aseptic conditions, following the 1:3 split ratio previously indicated for the shaking flask cultures (8.1 L of cell suspension+15.9 L of fresh media). Low serum containing media (DMEM/F12+1.25% FBS) is used for the cell growth in the production system (Wave Bag or 500 L bioreactor). 5% serum can be used for the cell growth in the Wave Bag if it is used as seed for the 500 L bioreactor.

C1F culture in Wave Bag is either harvested for production or used to inoculate a 500 L bioreactor.

### Culture in 500 L Bioreactor

The contents of the Wave Bag (25 L) are aseptically transferred to a large-scale bioreactor (total volume of 700 L with maximum working volume of 500 L) with 100 L of initial culture media (with a 1:3 to 1:6 split ratio to a total volume of 125 L).

After 3 days (+/−0.5 days) of culture, the media volume is increased to 500 L by the addition of 375 L new culture media and continued for an additional 3 days (+/−0.5 days). Cultures are sampled regularly to determine cell number and viability. Bioreactor culture is monitored off-line for pH, lactate, glucose, glutamine and glutamate levels.

### Concentration and Recovery

The cell culture broth is concentrated (25-100 fold) using a vertical axis flow through decanter centrifuge. The method for cell separation could include centrifugation, filtration, flocculation and combination thereof. The speed of the centrifuge is 500-1000 rcf with a flow rate per bowl size of 0.4-1.2 min⁻¹. The concentrated cell culture slurry is collected and moved to the next stage of washing process.

### Washing the Cells

The carryover of media components in the cultured meat is alleviated by efficiently washing the cell pellet after centrifugation. Specifically, the cell pellet obtained after centrifugation of the spent medium at the end of the cell culture is washed twice sequentially via a resuspension & centrifugation process using five-fold (w/v) 0.45% NaCl solution. By washing, the effective reduction of the media component carryover in the cultured meat is at least 25-fold. Except for glucose, glutamine & sodium, the carryover of the media components is empirically estimated to be very low, <10 ppm based on the 25-fold dilution at the end of washing. Glucose and glutamine are consumed as carbon/nitrogen sources during the cell culture.

The efficiency of washing is tracked by measuring the retained amount of Pluronic F-68 in the second wash solution. The initial concentration of the Pluronic F-68 in the growth media is 0.1% w/v (1000 mg/L). The Pluronic F-68 concentration in the second wash solution was not detectable (<<0.01% w/v) confirming the efficiency of washing in removing the other soluble media components.

Albumin in the wash solutions is detected and quantified using Bovine Albumin ELISA kit (Lifespan Biosciences) with high sensitivity and specificity for bovine serum albumin. In the final wash solution, the albumin concentration was determined to be lower than 10 mg/L and could be in the range of 0-100 ppm (mg/L)

The washed cells (Cultured Chicken) are stored in sealed, food-safe containers at less than or equal −20° C. prior to use for final product formulation.

### Example 3: Testing Safety of Cells for Bacteria and Viruses

Safety and efficacy of the cells is checked at all stages of growth and harvesting of the cells. Cultured C1F cells are evaluated for presence of viral, yeast, and bacterial adventitious agents.

The chicken product is analyzed for the presence of bacteria using protocols from the FDA's Bacteriological Analytical Manual (BAM).

Total Plate Count (TPC) is synonymous with Aerobic Plate Count (APC). As indicated in the US FDA's Bacteriological Analytical Manual (BAM), Chapter 3, the assay is intended to indicate the level of microorganism in a product. Briefly, the method involves appropriate decimal dilutions of the sample and plating onto non-selective media in agar plates. After incubating for approximately 48 hours, the colony forming units (CFUs) are counted and reported as total plate count.

Yeast and mold are analyzed according to methodology outlined in the US FDA Bacteriological Analytical Manual (BAM), Chapter 18. Briefly, the method involves serial dilutions of the sample in 0.1% peptone water and dispensing onto a petri plate that contains nutrients with antibiotics that inhibit microbial growth but facilitate yeast and mold enumeration. Plates are incubated at 25° C. and counted after 5 days. Alternately, yeast and mold are analyzed by using ten-fold serial dilutions of the sample in 0.1% peptone water and dispensing 1 mL onto Petrifilm that contains nutrients with antibiotics that facilitate yeast and mold enumeration. The Petrifilm is incubated for 48 hours incubated at 25 or 28° C. and the results are reported as CFUs.

Escherichia coli and coliform are analyzed according to methodology outlined in the US FDA Bacteriological Analytical Manual (BAM), Chapter 4. The method involves serial decimal dilutions in lauryl sulfate tryptone broth and incubated at 35° C. and checked for gas formation. Next step involves the transfer from gassing tubes (using a 3 mm loop) into BGLB broth and incubated at 35° C. for 48+/−2 hours. The results are reported as MPN (most probable number) coliform bacteria/g.

Streptococcus is analyzed using CMMEF method as described in chapter 9 of BAM. The assay principle is based on the detection of acid formation by Streptococcus and indicated by a color change from purple to yellow. KF Streptococcus agar medium is used with triphenyl tetrazolium chloride (TTC) for selective isolation and enumeration. The culture response is reported as CFUs after incubating aerobically at 35+/−2° C. for 46-48 hours.

Salmonella is analyzed according to methodology outlined in the US FDA Bacteriological Analytical Manual (BAM), Chapter 5. Briefly, the analyte is prepared for isolation of Salmonella then isolated by transferring to selective enrichment media, the plated onto bismuth sulfite (BS) agar, xylose lysine deoxycholate (XLD) agar, and Hektoen enteric (HE) agar. This step is repeated with transfer onto RV medium. Plates are incubated at 35° C. for 24+/−2 hours and examined for presence of colonies that may be Salmonella. Presumptive Salmonella are further tested through various methodology to observe biochemical and serological reactions of Salmonella according to the test/substrate used and result yielded. Quantities tested from 500 L harvests will be consistent with FDA BAM—Chapter 5.

Cultured chicken was prepared by methods consistent with the examples above. Table 1 indicates that bacteria contamination was negligible when compared to US FDA guidelines.

TABLE 1
Microbiological analysis of Cultured Chicken

| | | | | Representative | |
|---|---|---|---|---|---|
| Parameter | Basis Method | Specification | | Example | |
| Microbiological Analysis | | | | | |
| Aerobic plate count | FDA BAM-Chapter 3 | <10,000 | cfu/g | <10 | cfu/g |
| Coliforms | FDA BAM-Chapter 4 | <3 | MPN/g | <3 | MPN/g |
| E. coli | FDA BAM-Chapter 4 | <3 | MPN/g | <3 | MPN/g |
| Fecal Streptococcus | CMMEF-Chapter 9 | <10 | cfu/g | <10 | cfu/g |

| | | | |
|---|---|---|---|
| Salmonella | FDA BAM-Chapter 5 | Not Detected | Not Detected |

### Mycoplasma Contamination

Cultured C1F cells are considered valid for Mycoplasma detection if a minimum 3% of randomly selected and tested cell vials from each bank or 0.4× (square root of n), with n being the total number of cryovials banked with the animal cells for the cell bank being tested are thawed and their culture supernatants provide a negative result using the MycoAlert™ Mycoplasma Detection Kit. Following the kit guidelines, the tested samples are classified according to the ratio between Luminescence Reading B and Luminescence Reading A: Ratio<0.9 Negative for Mycoplasma; 0.9<Ratio<1.2 Borderline (required retesting of cells after 24 hours); Ratio>1.2 Mycoplasma contamination.

### Viral Assessment

Viral assessment was performed by analyzing adventitious human and avian virus and bacterial agents through an Infectious Disease Polymerase Chain Reaction (PCR) performed by a third-party (Charles River Research Animal Diagnostic Services)—Human Essential CLEAR Panel; Avian Virus and Bacteria Panel.

C1F from cell banks are considered valid for viral assessment if a minimum of 3% of independent cell vials from the tested bank or 0.4×\sqrt{n}, with n being the total number of cryovials banked with the animal cells for the cell bank being tested are thawed and their cell pellets provide a negative result for the full panel of adventitious agents.

Cultured C1F cells are considered approved for absence of adventitious avian and human viral and bacterial agents as the independent cell pellets from each cell bank were negative for the entire human and avian panels.

### Example 4: Cultured Chicken Analysis

The nutritional profile of Cultured Chicken was compared to conventional chicken.

A chemical analysis of Cultured Chicken was performed using moisture, protein content, fat content, ash content, carbohydrate. Moisture content was analyzed using the gravimetric oven drying method using a 10-gram test portion of the Cultured Chicken dried at 105° C. for >24 hours in a convection oven. The total crude protein was analyzed based the total nitrogen determined by Dumas combustion method using the LECO FP 628 Nitrogen/Protein Analyzer. The fat content was measured as cumulative fatty acid methyl esters (FAMEs) in ratio to the mass of the starting test portion. A 30 mg dried test portion of cells is subjected to direct transesterification by methanolic hydrochloric acid and FAMEs are separated for analysis by GC-FID by liquid-liquid extraction into heptane. Quantitation was achieved by addition of methyl-10-heptadecenoate as an internal standard added to test samples and the calibration standards. FAMEs identified in this method are constituents of GLC-74X analytical standard purchased from Nuchek Prep Inc., which is a mixture of 15 common saturated and unsaturated FAMEs between methyl octanoate and methyl docosanoate. All other significant peaks in the GC chromatograms were quantitated based on the calibration curve of their closest eluting neighbor. The total ash content was analyzed based on the gravimetric method by using the Milestone Pyro 260 Microwave Oven. The sample was heated to 900° C. for over 50 minutes and then held at 900° C. for 1 hour. The carbohydrate content is calculated by difference from the total of moisture, protein, ash, and fat content.

Table 2 summarizes the percent ash, carbohydrates, protein, and fat of Cultured Chicken compared to conventional boneless chicken breast.

TABLE 2
Nutritional analysis of Cultured Chicken in comparison with
conventional boneless chicken breast.

| | | | | Dry raw Cultured |
|---|---|---|---|---|
| | | Dry raw | Dry raw | Chicken |
| Nutritional | Method | chicken | Cultured | (normalized |
| package | reference | breast | Chicken | to 0% ash) |
| Ash | AOAC 930.30 | 0 | 12 | 0 |
| Carbohydrates | Calculation | 0 | 3 | 3.4 |
| Protein | AOAC 992.23 | 87.1 | 77.8 | 88.3 |
| Total Fat | AOAC 996.06 | 8.2 | 8.1 | 9.2 |

Table 3 summarizes the percent saturated, monounsaturated and polyunsaturated fats of cultured chicken compared to conventional boneless chicken breast. Fat values are presented as % of specific fat relative to total fat in the sample.

TABLE 3
Summary of the percent saturated, monounsaturated and polyunsaturated
fats of Cultured Chicken.

| Nutritional | Method | Dry raw | Dry raw JUST |
|---|---|---|---|
| package | reference | chicken breast | Cultured Chicken |
| Fat-Saturated | AOAC 996.06 | 26.1 | 36.8 |
| Fat-Monounsaturated | AOAC 996.06 | 34.1 | 50 |
| Fat-Polyunsaturated | AOAC 996.06 | 17.5 | 7.4 |
| Calories | Calculation | 437 | 436 |

The Cultured Chicken is similar to that of conventional chicken when comparing the grams per 100 gram of dry cell paste to dry raw chicken. The overall caloric value of dry conventional (farm raised) chicken breast and dry cultured chicken is similar. Monounsaturated fats (commonly referred to as the healthy type of fat) represent the type of fat in higher percentage in both conventional and Cultured Chicken (34.1% and 50%, respectively), followed by saturated fats and polyunsaturated fats. Interestingly, the high ash content in Cultured Chicken is due to residual salt, primarily from the 0.45% NaCl washes used to prepare the material, and from the culture medium used to grow the chicken cells. This was also confirmed by the sodium levels in Cultured Chicken (3.6%). When ash is removed from the analysis, protein, fat, and carbohydrate levels are quite consistent between Cultured Chicken and conventional chicken.

### Example 5: Avian Food Product Composition

A representative avian food product composition is described below (by weight percentage) in Table 4.

TABLE 4
Example avian food product composition.

| Ingredient | % by weight |
|---|---|
| Water | 20-40 |
| Cell paste | 25-50 |
| Mung bean | 10-20 |
| Fat | 5-20 |
| transglutaminase | 0.0001-0.1 |

### Example 6: Sequencing Analysis on the Chicken Cells Used for Manufacturing

Sequencing analysis on the chicken cells used for manufacturing was compared to the parental cells to evaluate potential genetic drift induced by the culture conditions.

Briefly, differential gene expression analysis was done using the R program DESeq2_1.20.0, based on the referenced publication. Afterwards, the hierarchical clustering of samples was performed with ClusterProfiler: cluster_2.0.7-1.

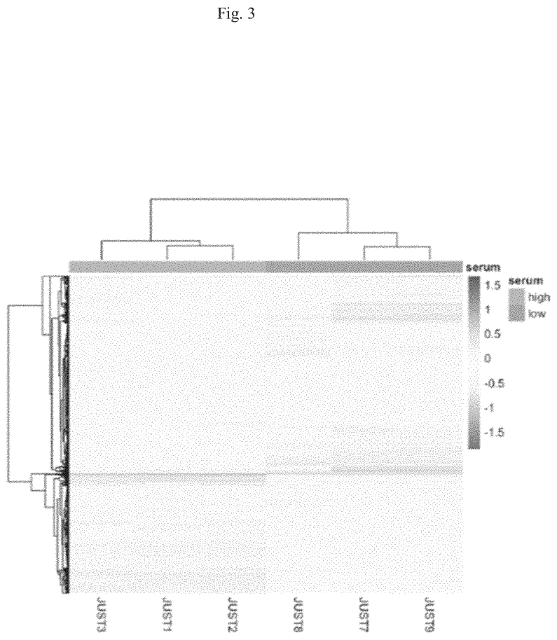

FIG. 3 depicts the clustering analysis performed between three biological replicates of parental chicken cell pools and three biological replicates of chicken cell pools used for manufacturing of Cultured Chicken.

More than 10,400 genes are plotted in FIG. 3, with statistically differently expressed genes selected for p<0.01, and the scale of differently expressed genes being presented as a heatmap.

Samples JUST1-JUST3 were obtained from parental chicken cells cultured in adherent conditions with media supplemented with high (10% v/v) serum concentration. Samples JUST 7-JUST9 were cultured in suspension with media supplemented with low (1.25% v/v) serum concentration. As observed in FIG. 3, samples clustered together within each group, demonstrating homogeneity between biological replicates within each culture condition.

Pathway enrichment was performed using enrichKEGG based on annotations on the Gallus gallus database (GenomeInfoDbData_1.1.0 and Org.Gg.eg.db (Gallus database) v2.1 updated Apr. 9, 2018), to verify if the differently expressed genes were grouped in certain pathways.

Pathways that were influenced include those associated with mechanisms of DNA replication, proteasome, ribosome, apoptosis and steroid biosynthesis. None of up- and down-regulated genes were associated with metabolites, proteins or other toxins harmful for human consumption.

### Example 7: Effect of Reducing Serum Content

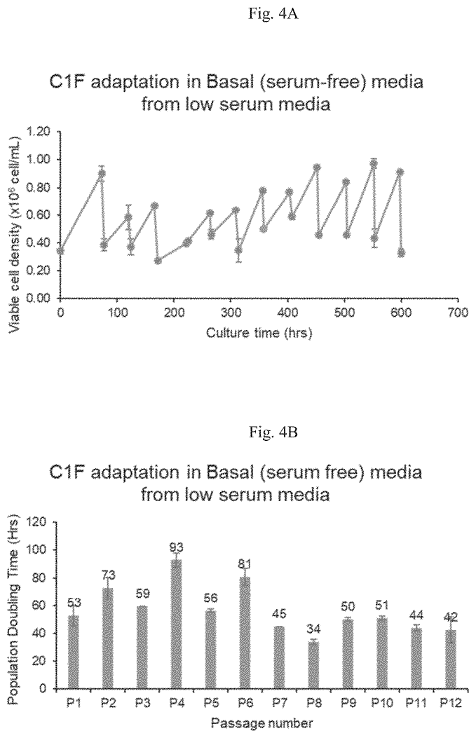

The effect of low serum media on cell viability (FIG. 4A) and population doubling time was analyzed (FIG. 4B). Cells were grown initially at 0.5% (v/v) serum concentration and then lowered to 0% (v/v)—serum-free.

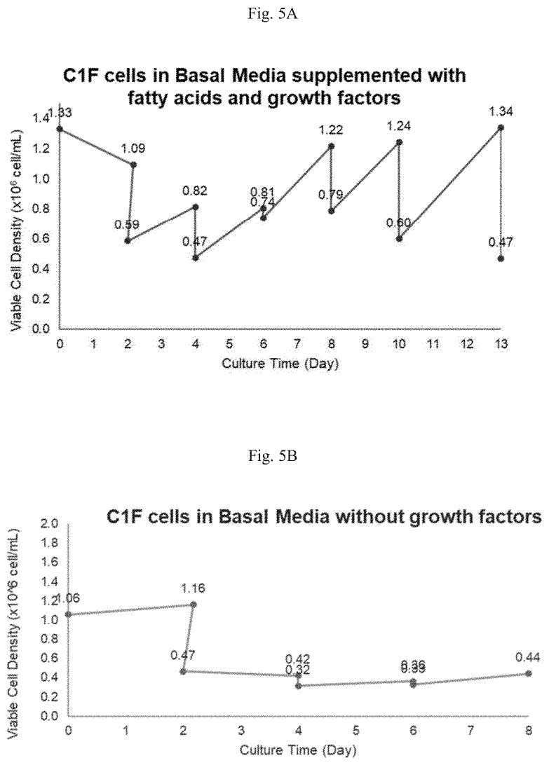

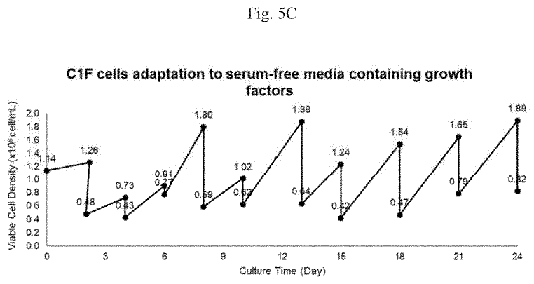

The effect of C1F cell growth in basal media with no serum that was supplemented with fatty acids and growth factors (FIG. 5A), and in basal media with no serum that was supplemented with fatty acids and growth factors (FIG. 5C) were studied and compared to C1F cells grown in basal media with no serum and without growth factors (FIG. 5B). The growth factors used were insulin-like, epidermal-like, and fibroblast-like growth factors at concentrations between 5-200 microgram/mL. FIGS. 5A and 5C used 100 microgram/mL of growth factors during experimentation. Similar effects were observed with growth factors at 50 microgram/mL. The results of which demonstrate that serum free media supplemented with growth factors achieve similar viable cell density as basal media with serum that is supplemented with growth factors.

### Example 8: Adaptation to Serum-Free Conditions

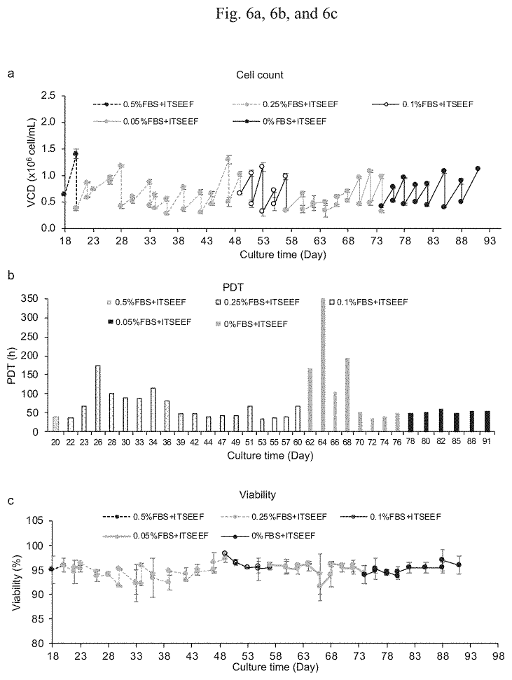

A methodology of gradual adaptation was implemented based on sequential reduction of serum percentage at each step, after assuring successful cell adaptation from the previous step. Cellular adaptation to lower serum concentration is not an immediate process and requires a period of time to get adjusted to the new microenvironment and to acquire a healthy appearance and an obvious growth at each stage of serum reduction. First, we determined the threshold concentration of FBS below which cells in suspension show significant growth arrest. C1F cells maintained in 5% FBS containing media were transferred to 2%, 1% and 0.5% FBS. When FBS concentration was reduced below 1% (v/v), cells showed of reduced growth. In order to adapt cells to low-serum concentration, media containing 1% v/v and 0.5% (v/v) FBS were supplemented with insulin-transferrin-selenium-ethanolamine (ITSE) (ThermoFisher) and growth factors (epidermal growth factor (EGF) and basic fibroblast growth factor (FGF), Peprotech). The use of ITSE, EGF and basic FGF together is referred to as ITSEEF. FIG. 6 discloses viability, population doubling time and population doubling level of cells adapted to grow in serum free media. FIG. 6a shows the viable cell density during the serum weaning process. FIG. 6b shows population doubling time during the serum weaning process. FIG. 6c shows the viability of C1F cells as the cells are transitioned from media containing 0.5% FBS to 0% FBS.

In order to achieve higher cell density in serum-free media, additional chemically defined supplements were tested. As shown in Table 5 (JUST basal media), vitamins, lipids, and trace elements were screened together with weaning of growth factors and ITSE. In this example, both powder (ThermoFisher, Cat #A42914EK) and liquid (basal media (DMEM/F-12, Cat #11320-033) supplemented with Pluronic-F68 and ITSEEF, so called JUST Basal (JB) media going forth) versions of DMEM/F12 media were used. Liquid DMEM/F12 was used for most of the adaptation study. SFM (SFC-2) with standard osmolarity (around 330 mOsm/Kg) was prepared using a commercially available powdered form of DMEM/F12 while SFM (SFC-4) with low-osmolarity (around 280 mOsm/Kg) was prepared using a custom-made variant of powder DMEM/F12 which did not contain glucose, HEPES buffer, L-glutamine, sodium bicarbonate, and sodium chloride. Missing components of SFC-4 were added separately and osmolarity was adjusted based on different values of sodium chloride addition. In-house RO/DI water was used to prepare DMEM/F12 basal media from powder formulations.

TABLE 5
Composition of the different SFM optimized at different stages
of adaptation to serum-free condition.

| | | SFM Type | | | |
|---|---|---|---|---|---|
| Components | | JB | JB-VLA | SFC-2 | SFC-4 |
| Basal media | DMEM/F12 | X | X | X | X |
| Protein-based | ITSE | X | X | X | X |
| Growth | EGF | X | X | | |
| Factors | Basic FGF | X | X | | |
| Vitamins | 4Vit Mix | | X | X | X |
| Lipids | CDL Mix | | X | X | X |
| Trace | Commercial Trace | | X | X | X |
| Elements | Element Mix | | | | |
| Surfactant | Pluronic | X | X | X | X |

### Serum-Free C1F (SF-C1F) Cell Expansion and Cryopreservation

Based on viable cell density, the split ratio for the expansion of C1F cells was determined, which is typically 1:3 (v/v). C1F cells cultured in serum-free media (SFM) were expanded from 125 mL flask with 50 mL working culture to a final step at 5 L flask with 2.5 L working volume, via multiple incremental subculture steps: 100 mL in 250 mL flasks, 300 mL in 500 mL flasks, 900 mL in 2.8 L flasks. After each cell passage, a new measurement of cell density and viability was done following the same protocol previously described.

SF-C1F cell banks were prepared from actively growing cultures in 5 L shake flask. The volume of C1F cell suspension that held the number of cells desired to bank was centrifuged at 300×g. The supernatant was aseptically decanted or aspirated without disturbing the C1F cell pellet. The cell pellet was gently resuspended in cryopreservation medium. Various in-house and commercially available freezing media were screened to determine the best performer (Table 6). In-house freezing media were prepared by adding FBS and/or DMSO to SFM (SFC-2) media. Commercial cryopreservation media were purchased from BioLife Solutions (CryoStor CS2, CS5, CS10) and PromoCell (Cryo-SFM). SF-C1F cell banks were stored as 20 to 30 million cell aliquots at −185° C. in the vapor phase of a liquid nitrogen freezer. One (1) mL aliquots for in-house cryopreservation media and 2 mL aliquots for commercial freezing media were dispensed into cryogenic storage vials. Cells were frozen in bar-coded cryovials at a rate of −1° C./min from 4° C. to −80° C. during a 16 to 24-hour period in isopropanol chambers. C1F cells were then transferred and stored in a vapor phase liquid nitrogen storage system (Taylor Wharton (<−175° C.)). Vial content and banked storage position were recorded in a controlled database. GMP chain of custody documentation (vial identity confirmation) is utilized to ensure the appropriate vial(s) are retrieved from the cell banks.

Two vials of SF-C1F cell bank were thawed in 37° C. water bath for less than 2 min and resuspended following 10-times dilution using SFM (SFC-2). After centrifugation at 300×g, the supernatant was removed, and the cells were resuspended again in fresh SFM at a density between 0.3-0.6×10⁶ cell/mL. Spin passage was carried out until the cells showed recovery by growing to a viable cell density (VCD) at or above 1.2×10⁶ cell/mL. Upon recovery cells were split passaged following 1:3 ratio. Spin passage was performed by centrifuging the cells at 300×g for 5 min and discarding the supernatant. The cell pellet was then resuspended in fresh medium. In the split passage method, a portion of cell culture was transferred to a new flask containing predetermined volume of fresh media. For a cell split ratio of 1:3, one third of the total volume of the original C1F suspension is transferred to a flask containing two thirds of total volume of fresh culture media. VCD was measured according to the method disclosed in Example 12.

TABLE 6
Cryopreservation media tested for SF-C1F cells.

| Media Type | Name/Components | Vendor | Type |
|---|---|---|---|
| Cryopreservation | 10% FBS + 10% DMSO + | In-house | Serum containing |
| media | 80% SFM | | |
| | 90% FBS + 10% DMSO | In-house | Serum containing |
| | 10% DMSO + 90% SFM | In-house | Serum-free |
| | CryoStor CS5 | BioLife Solutions | Serum-free |
| | CryoStor CS2 | BioLife Solutions | Serum-free |
| | CryoStor CS10 | BioLife Solutions | Serum-free |
| | Cyro-SFM | PromoCell | Serum-free |

For quantification of viable cell density and viability, 1 mL of C1F suspension were collected in an Eppendorf tube and centrifuged at 300×g for 5 min. The supernatant was discarded or used for determination of metabolite concentration. The C1F cell pellet was resuspended in 500 μL of TrypLE Express (Gibco) and incubated for 5-8 min at 37° C. on a shaking platform, followed by an inactivation of enzymatic activity by adding 500 μL of culture media. The total volume (minimum volume of 550 mL per sample) was transferred to sampling cups for the Vi-Cell™ XR Cell Viability Analyzer (Beckman Coulter). Cell density and viability was quantified using the Vi-Cell analyzer. Nova Flex bioanalyzer (Nova Biomedical, USA) was used to evaluate values of pH, glucose, glutamine, glutamate, lactate, ammonium, potassium, and sodium. One (1) mL of sample (spent or fresh media) was used for media component and metabolite analysis. The osmolarity of fresh and spent media was measured using OsmoPro osmometer (Advanced Instruments) using 20 μL of sample. Population doubling time (PDT) and Population doubling level (PDL) were calculated according to the following formulas:

PDT=t*log 10(2)/((log 10(n/n0)),

where t=culture time, n=final cell number and n0=number of cells seeded.

PDL=3.32[log 10(n/n0)],

where n=final cell number and n0=number of cells seeded.

After successful adaptation of the C1F cells to 0.5% FBS, FBS was further reduced in steps to 0.25%, 0.1%, 0.05% and to 0% FBS. C1F cells were successfully grown without FBS in the presence of ITSEEF, however, the cell density and proliferation rate was a little lower than cells grown in 5% FBS containing medium.

### Example 9: Additional Media Components

This example discloses the addition of nutritional components to serum free media to improve cell density and proliferation rate. The media disclosed in Table 5 is referred to as JUST Basal (JB) media. A lipid solution purchased from ThermoFisher (CD-lipid) was previously reported to aid in weaning of FBS in cell culture media. Lipids, especially essential fatty acids and ethanolamine have been shown to support increased growth of cells, including fibroblasts. They store energy and act as constituents of the cellular membrane; they also aid in signaling and transport. Supplementation of chemically-defined vitamins and lipids improved the VCD of serum-free C1F cells from about 0.8-1.0×10⁶ cell/mL to 1.5×10⁶ cell/mL. VCD was measured according to the method disclosed in Example 13.

Next, we added trace elements to increase VCD and proliferation rate. For instance, selenium is known to help detoxify free radicals as a cofactor for glutathione (GSH) synthetase. Other trace elements like copper, zinc and tricarboxylic acid are necessary albeit in small quantities for cell growth and proliferation. The micronutrients are also essential for the functionality and maintenance of certain enzymes. Trace elements A, B and C purchased from Corning were tested. Trace A mixture contains defined concentration of CuSO4, ZnSO4, Na-selenite, and ferric citrate. When cultured with Trace A (JB-VLA), C1F cells were able to achieve a VCD of ˜2×10⁶ cell/mL or higher over time. Interestingly Trace B and C had no observable effects on C1F chicken cells growth in SFM. VCD was measured according to the method disclosed in Example 12.

### Example 10: Reduction of Growth Factors

This example discloses the reduction of growth factors in SFM. C1F cells were cultivated as disclosed in Example 8 but were adapted to minimize the addition of growth factors by slowly reducing the amount of growth factors added to the media. Over time, C1F cells grew successfully at similar VCD and proliferation rates as disclosed in Example 8 in media that did not contain EGF and FGF.

Experiments to reduce ITSE were successful in reducing the amount of ITSE supplementation by 10-fold without compromising the growth and proliferation of chicken cells in SFM. VCD was measured according to the method disclosed in Example 12.

### Example 11: Large Scale Manufacturing of Avian Cells

Multiple large scale manufacturing of C1F cells in single use and stainless-steel bioreactors at scales of up to 1,000 L using serum-free (C1F-SFM) and serum-containing media (C1F-SCM) were performed. The serum-free and serum-containing media are described herein.

As the size of the fermentation vessel increases, high pressure, mixing time, nutrient flow, lower O₂ levels and buildup of CO₂, and shear act to inhibit or prevent growth of cells or lysis of the cells. As the size of the fermentation vessel increases in height, the pressure at the bottom of the vessel can be extremely high, leading to lysis of cells. It is a surprising and unexpected result that avian cells could be cultivated in large fermenters. Avian cells do not have a protective cell wall that protects the cell from high pressures.

Single use wavebag bioreactors were used in batch cell culture mode or perfusion mode.

For the batch cell culture mode, culture from 5 L shake flasks was used to inoculate a 50 L rocking motion wavebag under aseptic conditions, to obtain a desired split ratio. After a desired cell density was achieved, the additional media was added to the wavebag to achieve a desired split ratio. At this point the total culture volume was 50 L. Upon completion of the cultivation of the 50 L wavebag, the entire contents of two wavebag batch cultures were used to inoculate 200 L stainless steel bioreactor.

For the perfusion wavebag cultures used to generate inoculum for 500 L bioreactors, a single 50 L wavebag bioreactor was inoculated with culture from 5 L shake flasks and fresh media was added to achieve a desired split ratio. Following inoculation, the cell culture was allowed to grow for one day, before the perfusion process was initiated on day 1. Perfusion was continued for a predetermined amount of time and on the last day of the perfusion, the cell culture was transferred and used to inoculate the 500 L single use bioreactor following desired split ratio.

Multiple 200 L and 1,000 L stainless steel bioreactor cultivations were performed to manufacture cultured chicken cells. The contents of the wavebag culture discussed above were transferred to the 200 L bioreactor and culture media was added to the bioreactor to achieve a desired split ratio. During cultivation, the bioreactor culture was monitored off-line for pH, dissolved oxygen, glucose, glutamine, lactate, ammonium, and osmolarity levels. During the cultivation, samples were collected to confirm the absence of microorganisms.

The 1,000 L stainless steel and the 500 L single-use bioreactors may also be run in a draw and fill method. By this process, a desired amount of a bioreactor culture, for example 750 L of a 1,000 L bioreactor or 375 L of a 500 L bioreactor of culture are harvested into an interim storage container (single use BioBag) and fresh media is immediately added to the remaining culture, returning total volume to 1,000 L or 500 L. Concurrent with the refill operation, the collected cell culture is concentrated for harvesting purposes. Once the bioreactor has been refilled to its desired volume, cultivation was continued to achieve a desired cell density. The draw and fill procedure may be performed multiple times culminating with a final harvest collecting the full culture volume.

Cell harvest is defined as separation and collection of cells from growth media/liquid. Typically, the harvest is performed by centrifugation and washing of residual media components. The cells can be washed with any wash solution, typically water containing 0.45% (w/v) NaCl. The product of harvest, cultured chicken, is also termed “cell paste” which means wet cell pellet generated after centrifugation and washing.

Cell densities far exceeding 2 million cells/mL were routinely obtained.

### Example 12: Reducing Lactate Production

During cell growth, metabolite (e.g. lactate, ammonia, amino acid intermediates) accumulation have been shown to be detrimental to cell growth and productivity at certain concentrations (Claudia Altamirano et al., 2006; Freund & Croughan, 2018; Lao & Toth, 1997; Pereira et al., 2018). In a fed-batch process the accumulation of lactate causes a decrease in culture pH requiring the addition of alkali to maintain pH at setpoint or physiological range. Negatively, the addition of alkali causes an increase in the osmolality of the media and it has been shown that higher osmolality levels strongly inhibit the growth and protein production of most cell lines (Christoph Kuper et al., 2007; Kiehl et al., 2011; McNeil et al., 1999).

The major route of lactate accumulation is the interconversion of pyruvate to lactate which is catalyzed by lactate dehydrogenase (LDH). In mammalian cells, studies have shown that LDH exist either as homo- or hetero-tetramers with a subunit A or B, encoded by LDHA or LDHB respectively (Urbańska & Orzechowski, 2019). Moreover, it has been shown that LDHA catalyzes the forward reaction (pyruvate to lactate) and LDHB catalyzes the backward reaction (lactate to pyruvate). LDHA play a key role in the Warburg effect that occurs in cell lines that do not drive the breakdown of pyruvate through the citric acid cycle, producing lactate from pyruvate even in the presence of oxygen

Oxamate, an analogue of pyruvate, is a strong competitive LDHA inhibitor halting the Warburg effect by channeling much of the breakdown of glucose through the tricarboxylic acid (TCA) cycle—a much more energy efficient process (Wang et al., 2019). However, the use of this molecule inhibits cell proliferation which is a key factor at the earlier stage of production for most industrial mammalian cell lines (Kim et al., 2019; Wang et al., 2019).

C1F cells were cultivated in suspension culture supplemented with 1.25% bovine serum. Different concentrations of sodium oxamate were tested: 1, 3, 5, 10, 30, 60, 100, and 200 mM, and production of lactate, glucose consumption, cell growth rates and cell density were measured.

The specific rates were calculated using daily viable cell concentration and metabolite concentrations for the duration of the cell culture. Specific net growth rates (UN) were calculated as a change in VCD over a time interval t₁ to t₂ using equation (1):

$$μ N = \frac{I n [ \frac{VCD 2}{VCD 1} ]}{t 2 - t 1} \qquad ( Equation 1 )$$

Specific glucose consumption rate (qGluc) or specific lactate production rate (qLac) were determined using equation (2), where P is glucose or lactate concentration:

$$qGluc or qLac = μ N ( \frac{P 2 - P 1}{VCD 2 - VCD 1} ) \qquad ( Equation 2 )$$

Viable cell density (VCD) and viability were determined by the trypan blue exclusion method using the Vi-cell™ (Beckman Coulter) from 1 mL daily samples taken from shake flask cell culture. Gas and pH values including metabolite (glucose, lactate glutamine, glutamate, ammonium) concentrations were measured using the Bioprofile Flex analyzer (Nova Biomedical). Osmolality was measured using the OsmoPro Multi-Sample Micro-Osmometer (Advanced Instruments) which employs the freezing point technology.

C1F-SCM cells treated with different concentrations of sodium oxamate (1, 3, 5 and 10 mM), including untreated control cells, were cultured in a batch mode using duplicate shake flasks for 3 days. We observed a 28% (p<0.05) reduction in lactate production for 10 mM oxamate-treated cells and appreciable decrease in lactate production in cells treated with other concentrations of oxamate tested on day 2 compared to the control condition. In addition, we observed a concentration-dependent effect of oxamate on lactate production and glucose consumption by day 2 in oxamate-treated cells, with an increase in oxamate concentration leading to reduced lactate production. Other control parameters were within acceptable physiological ranges.

When the experiment was repeated using higher concentrations of sodium oxamate (10, 15, 20 and 30 mM) of sodium oxamate in a 3-day batch cell culture, lactate production was decreased in a concentration-dependent manner and lactate production was decreased by about 52%.

We went ahead to further increase the concentration (30, 60, 100 and 200 mM) of oxamate for a 3-day batch culture. As expected, a concentration-dependent decrease in lactate production in cells treated with oxamate was observed.

To further examine if the effects of oxamate remained similar in cell culture with metabolic by-products already present, we passaged cells treated with 30 mM oxamate using a 1:3 (v/v) split with fresh media (⅓ of the volume of spent media and ⅔ of fresh media). Having been used in culture for several days, carried over media invariably contains residual amounts (carryover) of metabolites resultant from cell consumption.

C1F cells treated with 30 mM of oxamate showed a continuous linear proliferation up to day 5 of culture, peaking at 2.79×10⁶ cells/mL. Control cultures peaked at day 3 of culture, at 1.64×10⁶ cells/mL and lagged after that. Hence, oxamate-treated culture showed a significant increase in maximum viable cell density of ˜44%. Even though the oxamate-treated cells exhibited a higher cell density by day 5, these C1F cells still had ˜23% reduction in cumulative lactate production compared to the control group. In addition, oxamate-treated C1F cells showed a decreased specific glucose consumption (qGluc). Cell viability and osmolality of the media were not compromised by the supplementation with oxamate. Ammonium accumulation spiked out for oxamate-treated cultures between days 3 and 5 of culture relative to the non-oxamate-treated control. The pH of the media ended up around 7.0 for the control cultures and 7.2 for oxamate-treated C1F cells.

The increase in cell density of oxamate treated cells is surprising and unexpected. Studies conducted using oxamate on cancer cells show inhibition of cell proliferation. The inhibitory effect of oxamate on cell proliferation may be due to the dependency of cancer cells on the glycolytic pathway as a source of energy as it represents a faster route for ATP generation than via the TCA cycle (Kim et al., 2019; Lu et al., 2014).

### Example 13: Alternative Sugars

We evaluated the impact of alternative sugars (mannose, fructose and galactose) on the growth and metabolism of an in-house C1F chicken cells grown in suspension cultures containing 1.25% FBS. Specific net growth rate (uN) and Specific glucose consumption rate (qGluc) or specific lactate production rate (qLac) were calculated according to equation 1 or 2 as disclosed in Example 12.

Suspension cultures as described herein were cultivated using 3 g/L of the respective sugars were added from day 0 and cultured in a batch mode up to day 3. On day 3 after sampling, an additional 3 g/L of each sugar was added to the respective flasks. By day 3, at the peak cell density, flasks that used glucose as carbon source had the highest viable cell density (˜2.805×10⁶ cells/mL), followed by flasks with mannose (˜2.40×10⁶ cells/mL), then fructose (1.935×10⁶ cells/mL) and lastly galactose (0.915×10⁶ cells/mL).

Though mannose-fed flasks had a lower overall lactate production by day 2, when normalized to day 3 VCD, lactate produced showed a slight increase by 1.7% compared to cells cultured with glucose.

Since we observed that the C1F cells could utilize fructose as a carbon source, we evaluated the effect of different starting concentration of fructose. In one experiment, 6 g/L of fructose was added to one set of duplicate flasks from day 0, and 3 g/L of fructose added each to another set of duplicate flasks from day 0. In the flasks starting with 3 g/L of fructose, an additional 3 g/L of fructose was added on day 1 of one of the duplicates and day 2 of the other duplicate. Overall, the flasks showed similar cell density and growth rate profiles by day 2, though flasks cultured with 6 g/L of fructose from day 0 showed a slight increase in lactate accumulation from day 1 to 36% higher by day 3.

We next evaluated the effect of combining glucose, mannose and fructose on growth and lactate production. Using a design-of-experiment (DOE) approach, 17 batch shake flask runs were carried out evaluating varying combinations of concentrations of glucose, mannose and fructose as energy sources for suspension chicken C1F cells. The experimental design used included 3 factors (glucose, mannose and fructose) and 4 levels (0, 0.5, 1.5 and 3.0 g/L). By day 3, cells with the base carbon sources 3.0 glucose/0.5 fructose/0.5 mannose had the highest viable cell density (VCD) of 3.8×10⁶ cell/mL, followed by 3.0 glucose/0.0 fructose/3.0 mannose and 3.0 glucose/3.0 fructose/3.0 mannose. In addition, the VCD of 3.0 glucose/3.0 fructose/3.0 mannose flasks increased from 3.54×10⁶ cell/mL to 3.78×10⁶ cell/mL by day 4. Interestingly, the above-mentioned flasks showed a similar lactate profile to the control flasks

To maximize VCD, DOE analysis showed the presence of glucose to be very significant (p value=0.001). This was followed by the presence of mannose. In addition, the DOE analysis showed that to maximize VCD with minimal lactate, glucose and mannose combinations needed to be optimized. Moreover, fructose combinations showed lowest lactate accumulation levels and lower VCDs. The cultures with lowest amount of glucose (or no glucose or low/no mannose) performed poorly compared to those with more glucose and certain amount of mannose. Meanwhile, three flasks (3 glucose/1.5 fructose/3 mannose, 3.0 glucose/0.0 fructose/3.0 mannose and 3.0 glucose/3.0 fructose/3.0 mannose) with 3.0 g/L of glucose and at least D 1.5 g/L mannose showed a slow consumption of glucose.

Since we discovered the importance of the presence glucose in a culture and the additional benefit of mannose in cell culture longevity, we screened different glucose/mannose ratios using DOE. The DOE design used included 2 factors (glucose and mannose) and 4 levels (0.5, 1.5, 3.0 and 4.0 g/L). By day 4 of cell culture we observed that flasks with 3.0 g/L of glucose with additional 1.5-3.0 g/L of mannose exhibited the highest VCDs (˜10-25% increase vs. control) and extended cell culture longevity compared to the control (only 3.0 g/L glucose). Flasks with 3.0 g/L glucose and 1.5 g/L mannose had a VCD of about 3×10⁶ cell/mL where control flasks with 3.0 g/L glucose and no mannose had a VCD of about 2.5×10⁶ cell/mL.

### Example 14: Wet Extrusion

A dough comprising 60-65% water, 10%-60% plant protein, 2-50% cultivated cells, and 0.01% to 2% of sodium chloride, a desired amount of a polysaccharide is prepared. An exemplary formula for the dough for wet extrusions is provided in table 7 below. The cultivated animal cells are blended into the dough to prepare the dough/cell admixture and loaded into the hopper. The dough/cell admixture is extruded by a twin screw extruder with a cooling die attached to the end of the extruder.

TABLE 7
Wet extrusion formula

| Ingredient | Percent |
|---|---|
| Wet cell mass of 5-15% cell by dry weight | 20-90% |
| Edible oil | 1-10% |
| Mung bean protein (60-100% protein) | 5-50% |
| Soy protein (60-100% protein) | 5-50% |
| Starch and/or fiber | 0-20% |
| Sugar and/or polysaccharides | 0-20% |
| Spices, flavorants, and/or odorants | 0-10% |
| Water as needed to bring moisture content of dough to 40-95% | As needed |

The dough is fed into a twin screw extruder at a rate of 1-10 kg/hour. Water is injected into the housing of the screw at 0.1 m downstream from the center point of the hopper at a rate of 0.5-10 kg/hour to produce a dough with a desired moisture content, for example 60%, and the screw speed is set to 200-2000 rpm. The temperature of the barrel is increased in a stepwise format to 70°, 100°, 130°, and 140° C. in the first four temperature zones of the barrel, respectively. The 5^{th} and 6^{th} temperature zones of the barrel is maintained at a temperature of 160° C. The dough from the barrel is fed into a cooled die to prepare the extrudate.

The extrudate is collected and stored in an airtight container, optionally in a freezer. Optionally, the extrudate can be dried using conventional or convection ovens or other drying methods to a desired moisture content. The extrudate is further processed by shredding, chopping, mixing, shaping, seasoning process to create a food product.

### Example 15: Dry Extrusion

A dough comprising 20-35% water, 10%-60% plant protein, 2-50% cultivated cells, and 0.01% to 2% of sodium chloride and a desired amount of a polysaccharide is prepared. An exemplary formula for the wet dough is provided in table 8 below. The cultivated animal cells are blended into the dough to prepare the dough/cell admixture and loaded into the hopper. The dough/cell admixture is extruded by a twin screw extruder with a cooling die attached to the end of the extruder.

TABLE 8
Dry extrusion formula

| Ingredient | Percent |
|---|---|
| Wet cell mass of 5-15% cell by dry weight | 20-70% |
| Edible oil | 1-10% |
| Mung bean protein (60-100% protein) | 5-50% |
| Soy protein (60-100% protein) | 5-50% |
| Starch and/or fiber | 0-20% |
| Sugar and/or polysaccharides | 0-20% |
| Spices, flavorants, and/or odorants | 0-10% |
| Water as needed to bring moisture content to 5-40% | As needed |

The dough is fed into a twin screw extruder at a rate of 1-10 kg/hour. Water is injected into the housing of the screw at 0.1 m downstream from the center point of the hopper at a rate of 0.5-10 kg/hour to produce a dough with a desired moisture content, for example 60%, and the screw speed is set to 200-2000 rpm. The temperature of the barrel is increased in a stepwise format to 70°, 100°, 130°, and 140° C. in the first four temperature zones of the barrel, respectively. The 5^{th} and 6^{th} temperature zones of the barrel is maintained at a temperature of 160° C. The dough from the barrel is fed into a cooled die to prepare the extrudate.

The extrudate is collected and stored in an airtight container, optionally in a freezer. Optionally, the extrudate can be dried using conventional or convection ovens or other drying methods to a desired moisture content. The extrudate is further processed by shredding, chopping, mixing, shaping, seasoning process to create a food product.

### Example 16: Rheological Properties of Cultivated Chicken Cells

The chicken and bovine cells of Example 11 cultivated in a 500 L or 1,000 L bioreactor were harvested, washed with water and dewatered to have a typical moisture content of about 88-94% water to wet produce cell paste. The chicken and bovine cells are approximately 70-80% water.

Rheological properties of cultured chicken and bovine cells were characterized with dynamic oscillatory rheology. A rheometer (Discovery Hybrid Rheometer, TA instruments) equipped with a flat parallel plate geometry (40 mm diameter) was used to monitor the viscoelastic properties of the cells upon exposure to increasing temperatures. Samples of the wet cell paste were measured as-is, without the addition of any other ingredients to determine the gelling and rheological properties of the cultivated cells. The harvested wet cell paste was 90.7% moisture, 7.1% protein, 0.8% fat 0.9% ash, and 0.5% carbohydrates. The cell density of the harvested wet cell paste ranged from 1×10⁹ to 1.25×10⁹/kg cell paste (1×10⁶ to 1.25×10⁶ cells per ml).

The rheological properties of a 7% mung bean protein solution were determined as a control.

About 1.5 mL of wet cell paste or the mung bean protein solution was loaded onto the lower plate of the rheometer and trimmed according to standard procedures. A solvent trap was loaded with 2 mL of distilled water was used to prevent evaporation of water within the sample during the measurement.

The storage (G′) and loss (G″) modulus were continuously recorded during a temperature ramp from 30° to 95° C. at a heating rate of 5° C./min under small deformation conditions (0.1% strain) at a constant angular frequency of 10 rad/s followed by a 1 minute hold at 95° C. After this hold, the temperature of the material was reduced to 50° C. and an amplitude sweep test from 0.01% to 100% strain was carried out at a constant frequency of 10 rad/s to characterize the gelled material's linear viscoelastic region. Each sample was run in duplicate.

Both chicken and beef wet cell paste showed good gelation ability as shown on data captured by Oscillation Temperature Ramp. Beef B4M-t6-S1 cells have an onset gelation temperature around 52° C. Chicken BR08-121818 cells have an onset gelation temperature around 65° C. Both the gels formed by beef and chicken cells are soft elastic gels with low stiffness. As the oscillation strain increased, the storage modulus decreased gradually instead of dropping sharply.

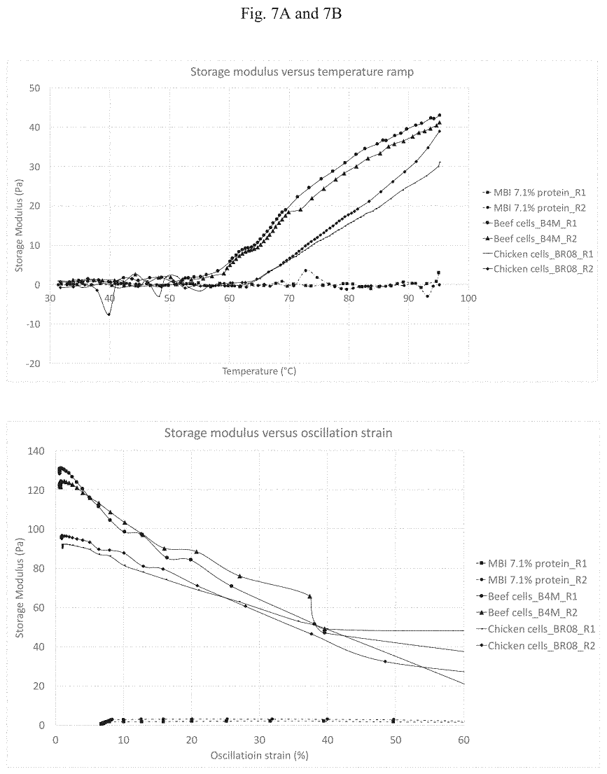

As shown in FIG. 7A, the storage modulus of the wet chicken cell remains constant as the temperature is raised until around 65° C. At 65° C., the storage modulus of the wet chicken cell paste starts to increase showing that the wet chicken cell paste is beginning to gel. Similarly, the storage modulus of wet bovine cell paste remains constant as the temperature is raised until around 52° C. At 52° C., the storage modulus of the wet bovine cell paste starts to increase showing that the wet bovine cell paste is beginning to gel. The 7% mung bean protein isolate control does not gel even at 95° C. as can be seen by the unchanging storage modulus.

FIG. 7B shows the impact on the storage modulus in response to increasing oscillation strain. The experiment was performed at 50° C. FIG. 7B shows that under increasing oscillation strain, the storage modulus decreases. Under low oscillation strain of 10%, the storage modulus of the wet chicken cell paste is about 85 Pa and under high oscillation of 60%, the storage modulus decreases to about 25 Pa. Under low oscillation strain of 10%, the storage modulus of the wet bovine cell paste is about 125 Pa and under high oscillation of 60%, the storage modulus decreases to about 25 Pa.

### Example 17: Extrusion of Cultivated Chicken Cells

The chicken cells of Example 11 cultivated in a 500 L or 1000 L bioreactor were harvested and a wet cell paste as described Example 13 was prepared and frozen. 250 g or the wet chicken cell paste was thawed and loaded into the liquid feed reservoir of the extruder. This chicken cell paste was then pumped by a peristaltic pump into the extruder barrel via the feeding probe. The feeding rate for cell paste was maintained at a constant rate during the extrusion process.

Dry ingredients, including soy protein, wheat protein, and table salt, were evenly mixed and transferred to the hopper of the dry feeder. The feeding rate for dry ingredient was maintained at a constant rate during the extrusion process.

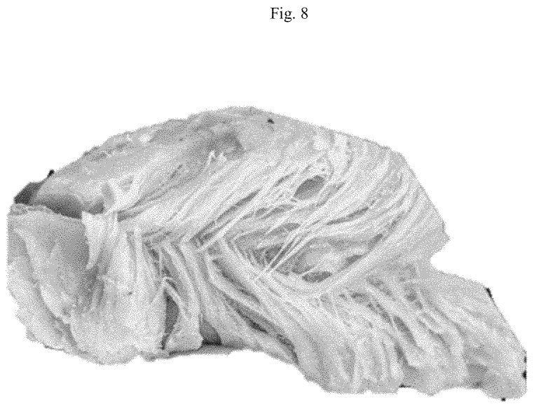

A photograph of a chicken cell extrudate containing 65% cultivated chicken cell paste is shown in FIG. 8. The fibrous texture of the extrudate is very similar to the fibrous texture of chicken breast from a farm raised chicken.

### Example 18: Texture Analysis of Cultivated Chicken Cell Extrudate

The texture of the extrudate of Example 14 was determined and compared to a cooked chicken breast of a farm raised chicken.

Chicken breast from a farm raised chicken was purchased from local supermarket. The chicken breast was vacuum packaged and sous vide cooked at 70° C. for 2 hr. This cooking condition is referred by many recipes to yield the most desirable tenderness. The extrudate of Example 14 containing 65% cultured chicken muscle cells, 20% soy protein concentrate, 14% wheat protein, 0.05% NaCl was collected as-is after extrusion process, without post-extrusion seasoning and shaping. A control plant-based extrudate containing plant protein isolates and no animal cells was produced using the same extrusion parameters. This plant-based sample contained 65% water, 20% soy protein concentrate, 14% wheat protein, 0.05% NaCl.

The above samples were allowed to cool down to room temperature before being expelled out of the syringes. All samples were sliced into 8 mm×15 mm×35 mm cubes for texture analysis on a TA.XTplus Texture analyser manufactured by Stable Micro Systems.

Instrumental texture profile parameters were recorded on the TA.XTplus Texture Analyser with a TA-65 multipuncture probe. This puncture probe utilizes 13 needles to puncture through sample and the hardness is defined as the total force received by all 13 needles during puncturing. Samples were submitted to a puncture test at a test speed of 3 mm/s for puncturing the sample to a total depth of 4 mm. The hardness of sample was defined as the maximum force reached during puncture

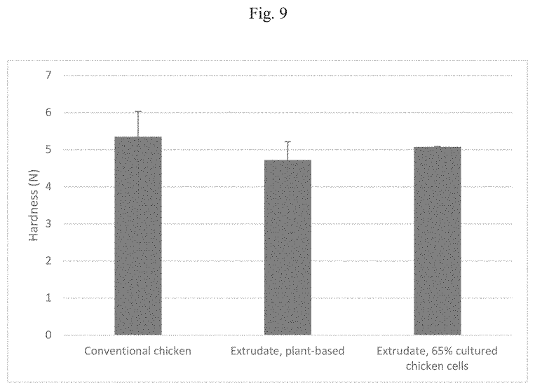

FIG. 9 shows that the hardness of the chicken cell extrudate was about 5 N, similar to the conventional chicken breast. The control plant based extrudate that did not contain cultivated chicken cells showed a lower hardness.

### REFERENCES

Lawler A & Adler J. 2012. Smithsonian Magazine. June. Available at http://www.smithsonianmag.com/history/how-the-chicken-conquered-the-world-87583657/.
USDA Fact Sheets—Poultry Preparation. Focus on: Chicken. Available at http://www.fsis.usda.gov/Fact_Sheets/Chicken_Food_Safety Focus/index.asp.
Gorman J. 2016. Chickens Weren't Always Dinner for Humans. NY Times. Jan. 18,2016. Available at www.nytimes.com/2016/01/19/science/chickens-werent-always-dinner-for-humans.html.
English D R, MacInnis R J, Hodge A M, Hopper J L, Haydon A M, Giles G G. 2004. Red meat, chicken, and fish consumption and risk of colorectal cancer. Cancer Epidemiology and Prevention Biomarkers. 13(9):1509-14.
Sinha R, Cross A J, Graubard B I, Leitzmann M F, Schatzkin A. 2009. Meat intake and mortality: a prospective study of over half a million people. Archives of internal medicine. 169(6):562-71; Hu F B, Rimm E B, Stampfer M J, Ascherio A, Spiegelman D, Willett W C. 2000. Prospective study of major dietary patterns and risk of coronary heart disease in men-. The American journal of clinical nutrition. 72(4):912-21.
International Agency for Research on Cancer (IARC). 2018. Monographs on the Evaluation of Carcinogenic Risks to Humans. Volume 114. Red Meat and Processed Meat. IARC, Lyon, France.
Physicians Committee for Responsible Medicine (PCRM). 2013. Letter to The Honorable Sanford Bishop, U S Congress, dated Mar. 14, 2013.
Altamirano, Claudia, Illanes, A., Becerra, S., Cairó, J. J., & Gòdia, F. (2006). Considerations on the lactate consumption by CHO cells in the presence of galactose. Journal of Biotechnology, 125(4), 547-556. https://doi.org/10.1016/j.jbiotec.2006.03.023
Freund, N. W., & Croughan, M. S. (2018). A simple method to reduce both lactic acid and ammonium production in industrial animal cell culture. In International Journal of Molecular Sciences (Vol. 19, Issue 2). https://doi.org/10.3390/ijms19020385
Lao, M. S., & Toth, D. (1997). Effects of ammonium and lactate on growth and metabolism of a recombinant Chinese hamster ovary cell culture. In Biotechnology Progress (Vol. 13, Issue 5, pp. 688-691). https://doi.org/10.1021/bp9602360
Pereira, S., Kildegaard, H. F., & Andersen, M. R. (2018). Impact of CHO Metabolism on Cell Growth and Protein Production: An Overview of Toxic and Inhibiting Metabolites and Nutrients. Biotechnology Journal, 13(3), 1-13. https://doi.org/10.1002/biot.201700499
Christoph Kuper, Franz-X. Beck, & Wolfgang Neuhofer. (2007). Osmoadaptation of Mammalian Cells—An Orchestrated Network of Protective Genes. Current Genomics, 8(4), 209-218. https://doi.org/10.2174/138920207781386979
Kiehl, T. R., Shen, D., Khattak, S. F., Jian Li, Z., & Sharfstein, S. T. (2011). Observations of cell size dynamics under osmotic stress. Cytometry Part A, 79 A(7), 560-569. https://doi.org/10.1002/cyto.a.21076
McNeil, S. D., Nuccio, M. L., & Hanson, A. D. (1999). Betaines and related osmoprotectants. Targets for metabolic engineering of stress resistance. Plant Physiology, 120(4), 945-949. https://doi.org/10.1104/pp. 120.4.945
Urbańska, K., & Orzechowski, A. (2019). Unappreciated role of LDHA and LDHB to control apoptosis and autophagy in tumor cells. International Journal of Molecular Sciences, 20(9), 1-15. https://doi.org/10.3390/ijms20092085
Wang, Z., Nielsen, P. M., Laustsen, C., & Bertelsen, L. B. (2019). Metabolic consequences of lactate dehydrogenase inhibition by oxamate in hyperglycemic proximal tubular cells. Experimental Cell Research, 378(1), 51-56. https://doi.org/10.1016/j.yexcr.2019.03.001
Kim, E. Y., Chung, T. W., Han, C. W., Park, S. Y., Park, K. H., Jang, S. B., & Ha, K. T. (2019). A Novel Lactate Dehydrogenase Inhibitor, 1-(Phenylseleno)-4-(Trifluoromethyl) Benzene, Suppresses Tumor Growth through Apoptotic Cell Death. Scientific Reports, 9(1), 1-12. https://doi.org/10.1038/s41598-019-40617-3
Lu, Q. Y., Zhang, L., Yee, J. K., Go, V. L. W., & Lee, W. N. (2014). Metabolic consequences of LDHA inhibition by epigallocatechin gallate and oxamate in MIA PaCa-2 pancreatic cancer cells. Metabolomics, 11(1), 71-80. https://doi.org/10.1007/s11306-014-0672-8

1. An extrudate comprising cultivated animal cells, a plant protein, at least one other ingredient, and optionally, a peptide cross-linking enzyme.

2. The extrudate of claim 1, wherein the cultivated animal cell is selected from the group consisting of an avian cell, a bovine cell, a porcine cell, and a seafood cell.

3. (canceled)

4. The extrudate of claim 2, wherein the cultivated animal cell is of the genus Gallus, Meleagris, Anas, Bos, or Sus.

5. The extrudate of claim 1, wherein the extrudate comprises cultivated animal cells in an amount that is between 1% to 90% by dry weight.

6. (canceled)

7. The extrudate of claim 1, wherein the plant protein is a plant protein isolate or a plant protein concentrate.

8. (canceled)

9. The extrudate of claim 1, wherein the plant protein is a pulse protein.

10. (canceled)

11. The extrudate of claim 9, wherein the pulse protein is obtained from mung beans (Vigna radiata).

12.-18. (canceled)

19. The extrudate of claim 1, wherein the at least one other ingredient is selected from the group consisting lipid, salt, sugar, fiber, humectant, flavorant, colorant, and preservative.

20. The extrudate of claim 19, wherein the composition of the lipid is a free fatty acid, triacylglycerol, or sterol.

21. (canceled)

22. (canceled)

23. The extrudate of claim 19, wherein the lipid is a plant oil selected from the group consisting of algal oil, canola oil, coconut oil, olive oil, palm oil, palm kernel oil, peanut oil, rice bran oil, safflower oil, soybean oil, sunflower oil, and mixtures thereof.

24. (canceled)

25. The extrudate of claim 19, wherein the salt is selected from the group consisting of sodium chloride, ammonium sulfate, ammonium phosphate, ammonium chloride, potassium chloride, potassium sulfate, or potassium phosphate, and a phosphate salt.

26.-43. (canceled)

44. A method of preparing an extrudate comprising cultivated animal cells, a plant protein, at least one other ingredient, and optionally, a peptide cross-linking enzyme, the method comprising the steps of:
a) loading dry plant protein, and optionally loading at least one other ingredient into the hopper of an extrusion machine to prepare a dry ingredient mixture;
b) placing the dry ingredient mixture into the barrel of the extrusion machine;
c) preparing a dough in the barrel by injecting a liquid into the barrel;
d) injecting cultivated animal cells into the barrel during conveyance of the dough or the dry ingredient mixture to prepare dough/cell admixture;
e) conveying the dough/cell admixture through the barrel under mechanical pressure, optionally heating the dough/cell admixture in the barrel; and
f) extruding the dough/cell admixture through a die to produce the extrudate.

45. (canceled)

46. The method of claim 44, wherein the extrusion method is a wet extrusion method or a dry extrusion method.

47. The method of claim 44, wherein the extrusion machine is a single screw extrusion machine or a twin screw extrusion machine.

48. (canceled)

49. The method of claim 44, wherein the cultivated animal cells are frozen or dried.

50. The method of claim 44, wherein the cultivated animal cells are cultivated in a suspension culture or an adherent culture.

51. The method of claim 50, wherein the cultivated animal cells are cultivated in a growth medium that comprises animal serum.

52. The method of claim 50, wherein the cultivated animal cells are cultivated in a growth medium that does not comprise animal serum.

53. A wet cell paste comprising cultivated animal cells, wherein when the cell density of the wet cell paste is between 1×10⁶ to 10×10⁶ cells per ml, at a temperature of between 30° C. and 95° C., the storage modulus (G′) is between 5 Pa and 300 Pa.

54. The wet cell paste of claim 53, wherein the cultivated animal cell is an avian cell, a bovine cell, a porcine cell, or a seafood cell.

55. (canceled)

56. The wet cell paste of claim 53, wherein the cultivated animal cell is of the genus Gallus, Meleagris, Anas, Bos, or Sus.

57. (canceled)

58. (canceled)

59. The wet cell paste of claim 53, wherein the wet cell paste has a gelation temperature of between 35° and 95° C.

60.-67. (canceled)
